(19) United States
(12) Patent Application Publication
(10) Pub. No.: US 2002/0098519 A1
(43) Pub. Date: Jul. 25, 2002

(54) Prediction of unknown biological function of the active site in proteins or/and polynucleotides, and its utilization

(75) Inventor: Naganori Numao, Tokyo (JP)

Correspondence Address: CONNOLLY BOVE LODGE & HUTZ LLP, SUITE 800, 1990 M STREET NW, WASHINGTON, DC, 20036-3425, US

(73) Assignee: Biofrontier Institue Inc., Kanagawa (JP)

(21) Appl. No.: 10/026,698

(22) Filed: Dec. 27, 2001

Related U.S. Application Data
(63) Continuation-in-part of application No. 09/899,030, filed on Jul. 6, 2001.

(30) Foreign Application Priority Data
Jul. 7, 2000 (JP) 2000-206129

(51) Int. Cl. G01N 33/53; G06F 19/00; G01N 33/48; G01N 33/50

(52) U.S. Cl. 435/7.1; 702/19

(57) ABSTRACT

Biological•functional activity or binding partner of an arbitrary amino acid sequence (or nucleotide sequence) is efficiently predicted by giving EIIP index values to the total amino acid sequence or nucleotide sequence of a natural-type or non-natural-type arbitrary protein, and comparing the frequency spectra obtained by subjecting the resulting EIIP sequences to DFT.

### CROSS REFERENCE TO RELATED APPLICATIONS

This is a continuation-in-part application of application Ser. No. 09/889,030, filed Jul. 6, 2001.

This application is based upon and claims priority of Japanese Patent Application No. 2000-206129, filed on Jul. 7, 2000, the contents being incorporated herein by reference.

### BACKGROUND OF THE INVENTION

1. Field of the Invention

The present invention relates to an intellectual information technology for efficiently predicting a novel biological•functional activity or bonding partner of an arbitrary protein (or nucleotide sequence).

2. Description of the Related Art

As methods for predicting the biological•functional activity of a protein (or nucleotide sequence), there are developed a number of methods dependent on the homology of amino acid sequences (or nucleotide sequences), but these methods are individual and lacks generality. Therefore, it is extremely difficult to predict the biological•functional activity in the case that the homology between sequences to be compared is low. At present, it is strongly demanded by society to develop a method which is convenient and has a high generality. In particular, it is strongly desired to provide a novel methodology for elucidating the function of the proteins (or nucleotide sequences) currently obtainable by genome analysis.

At present, in the case of predicting the biological•functional activity of an arbitrary amino acid sequence (or nucleotide sequence), most frequently employed method is a search for a motif present in the amino acid sequence (or nucleotide sequence) (A. Bairoch et al., Nucleic Acids Res., 20, 2019-2022 (1992)). This method is known as one method for efficiently predicting a function of an arbitrary protein (M. J. E. Sternberg, CABIOS, 7, 257-260 (1991)). And, another searching method is a homology search between the total amino acid sequences of an arbitrary novel protein and that of a protein whose biological activity is already known (R. F. Doolittle et al, Nature, 307, 558-560 (1984)). Heretofore, these two methods are the methods for predicting a function which are most frequently employed but they are individual and lack generality. Namely, when a motif or homology between the amino acid sequences of two proteins to be compared is not found out, it is impossible to predict biological•functional activity. It is also well known that a biological activity is not necessarily the same even when the amino acid sequences have a high homology. The improvement of the methods is still desired.

In 1985, Veljkovic et al reported an epoch-making methodology wherein, when EIIP index values are given to the amino acids (or nucleotide residues) of the total amino acid sequences (or nucleotide sequences) of at least two proteins having the same biological activity and the values are calculated according to a digital processing method, the frequency values of those proteins converge at a specific value regardless of homology (V. Veljkovic et al., IEEE 32, 337-341 (1985); V. Veljkovic et al., Cancer Biochem. Biophys., 9, 139-148 (1987)). This method may be useful as one classifying method for organic polymeric compound (proteins and nucleotides) having the same biological activity. They have heretofore reported much on characteristic frequency values of proteins (V. Veljkovic et al., Cancer Biochem. Biophys., 9, 139-148 (1987); I. Cosic, IEEE, 41, 1101-1114 (1994)), but they have not at all mentioned prediction of function-activity of protein alone and any specific intermolecular interaction. For solving these problems, it is necessary to extract specific frequency spectra derived from biologically-functionally active sites of an arbitrary amino acid sequence (or nucleotide sequence) according to a novel method.

The present inventor has already reported a general method for predicting an active site (a substrate-binding site or catalytically active site) of a protein (N. Numao et al., Biol. Pharm. Bull., 16, 1160-1163 (1993)). That is, it is reported that at least one of 13 kinds of complementary amino acid units [GT, AS, GA, ID, TR, SR, LK, TXW, VXH, MXH, WXP, AXC, GXS (wherein G, T, A, S, I, D, R, L, K, W, V, H, M, P, C, and X mean glycine, threonine, alanine, serine, isoleucine, aspartic acid, arginine, leucine, lysine, tryptophan, valine, histidine, methionine, proline, cysteine, and any of 20 kinds of amino acids, respectively)] or complementary amino acid units of reverse sequences thereof is present in an active site region of an arbitrary protein. Furthermore, the present inventor has reported that, in the case of predicting a catalytically active region of a nucleotide sequence such as a ribozyme, it is the active region where an above motif sequence is present in a translated hypothetical amino acid sequence (N. Numao et al, EP Appl. No. 91311129.0 (1991)). Although the method is useful as a method for predicting an active site of the amino acid sequence or nucleotide sequence of any kind of proteins, it does not clarify any relevancy between kind of biological•functional activity and active site region and any specific intermolecular interaction. For solving the problems, it is necessary to give a physical constant to an amino acid sequence (or nucleotide sequence) which takes part in intermolecular interaction and carry out a mathematical analysis.

### SUMMARY OF THE INVENTION

Object of the present invention is to provide a method for predicting biological•functional activity derived from an active site of a protein (or nucleotide sequence) more efficiently with higher generality as compared with conventional methods.

Specifically, the present invention provides a method for predicting biological•functional activity (or binding activity) of desired arbitrary protein and/or nucleotide sequence, employing:

1) a total amino acid sequence frequency spectrum obtained by giving EIIP (Electron-ion interaction potential) index values to the amino acid residues of an arbitrary amino acid sequence and subjecting the resulting numerical value sequence (hereinafter, referred to as “EIIP sequence”) to discrete Fourier transformation (hereinafter, referred to as “DFT”), and/or

2) a frequency spectrum (hereinafter, referred to as “active site frequency spectrum”) obtained by giving EIIP index values to the amino acids of an amino acid sequence region, which is composed of 2 to 64 amino acid residues present in the arbitrary amino acid sequence and containing at least one known motif pertinent to an active site and subjecting the resulting EIIP sequence to DFT, and/or

3) a total nucleotide sequence frequency spectrum obtained by giving EIIP index values to the nucleotide residues of a nucleotide sequence region academically corresponding to the arbitrary amino acid sequence and subjecting the resulting EIIP sequence to DFT, and/or

4) a total nucleotide sequence frequency spectrum obtained by giving EIIP index values to the nucleotide residues of an arbitrary single-strand nucleotide sequence and subjecting the resulting EIIP sequence to DFT, and/or

5) a total nucleotide sequence frequency spectrum obtained by giving EIIP index values to the nucleotide residues of a nucleotide sequence which binds to a arbitrary single-strand nucleotide sequence through hydrogen bonding, and subjecting the resulting EIIP sequence to DFT,

the arbitrary amino acid sequence or nucleotide sequence being originated in natural-type or non-natural-type. The invention aims at facile prediction of a novel biological•functional activity hitherto unknown, such as decarboxylation activity toward an oxaloacetate of a prion protein and a β-amyloid precursor, similar biological activity between calcitonin and human growth hormone, or binding of Ebola virus to 55kd TNF receptor.

The method of the present invention for predicting biological•functional activity and/or binding activity of an arbitrary protein is a method for predicting biological•functional activity and/or binding activity of an arbitrary protein, which comprises:

determining a total amino acid sequence frequency spectrum obtained by giving EIIP (Electron-ion interaction potential) index values to the amino acid residues of an arbitrary amino acid sequence originated in natural-type or non-natural-type and subjecting the resulting EIIP sequence to DFT and

an active site frequency spectrum obtained by giving EIIP index values to the amino acids of an amino acid sequence region, which is composed of 2 to 64 amino acid residues present in the above arbitrary amino acid sequence and contains at least one known motif pertinent to an active site and subjecting the resulting EIIP sequence to DFT; and

selecting one or more characteristic frequency values derived from an active site of the protein from the cross-spectrum of the above total amino acid sequence frequency spectrum and the above active site frequency spectrum, and searching for one or more approximate frequency values of well-known characterized proteins similar to the characteristic frequency values described above.

In one embodiment of the above method of the present invention, as the known motif as a signal of the active site, any one or more of GT, AS, GA, ID, TR, SR, LK, TXW, VXH, MXH, WXP, AXC, GXS (wherein G, T, A, S, I, D, R, L, K, W, V, H, M, P, C, and X mean glycine, threonine, alanine, serine, isoleucine, aspartic acid, arginine, leucine, lysine, tryptophan, valine, histidine, methionine, proline, cysteine, and any of 20 kinds of amino acids, respectively) and/or reverse sequences thereof are employed.

The method of the present invention for predicting biological•functional activity and/or binding activity of an arbitrary protein is a method for predicting biological•functional activity and/or binding activity of an arbitrary protein, which comprises:

determining a total amino acid sequence frequency spectrum obtained by giving EIIP (Electron-ion interaction potential) index values to the amino acid residues of an arbitrary amino acid sequence originated in natural-type or non-natural-type and subjecting the resulting EIIP sequence to DFT and

a total nucleotide sequence frequency spectrum obtained by giving EIIP index values to the nucleotide residues of a nucleotide sequence region academically (J. C. Lacey, Chemtracts Biochem. Mol. Biol., 12, 398-418 (1999); V. Kunin, Orig. Life Evol. Biosphere 30, 459-466 (2000)) corresponding to the above amino acid sequence and subjecting the resulting EIIP sequence to DFT; and

selecting one or more characteristic frequency values derived from the protein from the cross-spectrum of the above total amino acid sequence frequency spectrum and the above active site frequency spectrum, and searching for one or more approximate frequency values of well-known characterized proteins similar to the characteristic frequency values described above.

The method of the present invention for predicting biological•functional activity and/or binding activity of an arbitrary protein is a method for predicting biological•functional activity and/or binding activity of an arbitrary nucleotide sequence, which comprises:

determining first total nucleotide sequence frequency spectrum obtained by giving EIIP index values to the nucleotide residues of an arbitrary single-strand nucleotide sequence originated in natural-type or non-natural-type and subjecting the resulting EIIP sequence to DFT and

second total nucleotide sequence frequency spectrum obtained by giving EIIP index values to the nucleotide residues of a complementary nucleotide sequence which binds to the above nucleotide sequence through hydrogen bonding, and subjecting the resulting EIIP sequence to DFT; and

selecting one or more characteristic frequency values derived from the nucleotide sequence from the cross-spectrum of the above first total nucleotide sequence frequency spectrum and the above second nucleotide sequence frequency spectrum, and searching for one or more approximate frequency values of well-known characterized proteins similar to the characteristic frequency values described above.

Moreover, the present invention relates to a method for predicting biological•functional activity and/or binding activity of an arbitrary amino acid sequence originated in natural-type or non-natural-type and other nucleotide sequence by comparing with each spectrum.

Furthermore, the present invention relates to a method for predicting an active site of an arbitrary amino acid sequence or nucleotide sequence originated in natural-type or non-natural-type by comparing with each spectrum.

### BRIEF DESCRIPTION OF THE DRAWINGS

FIGS. 1A to 1G are drawings illustrating examples of carrying out operations of the present invention.

FIG. 2 is a drawing illustrating a self-cross-spectrum of a magainin 2 precursor.

FIG. 3 is a drawing illustrating a cross-spectrum between a magainin 2 precursor and magainin 2.

FIG. 4 is a drawing illustrating a cross-spectrum between a magainin 2 precursor and a magainin 2 precursor wherein the amino acids of 221 to 233 are replaced by leucine.

FIG. 5 is a drawing illustrating a mixed frequency spectrum of a magainin 2 precursor.

FIG. 6 is a drawing illustrating a mixed frequency spectrum of magainin 2.

FIG. 7 is a drawing illustrating a mixed frequency spectrum of MSI-78A.

FIG. 8 is a drawing illustrating a self-cross-spectrum of a salmon calcitonin precursor.

FIG. 9 is a drawing illustrating a cross-spectrum between a salmon calcitonin precursor and salmon calcitonin.

FIG. 10 is a drawing illustrating a cross-spectrum between a salmon calcitonin precursor and the salmon calcitonin precursor wherein the amino acids of 83 to 114 are replaced by leucine.

FIG. 11 is a drawing illustrating a cross-spectrum between a salmon calcitonin precursor and the salmon calcitonin precursor wherein the amino acids in the region other than the region at 83 to 114 are replaced by leucine.

FIG. 12 is a drawing illustrating a self-cross-spectrum of gamma interferon.

FIG. 13 is a drawing illustrating a cross-spectrum between gamma interferon and an active site region (132 to 162) thereof.

FIG. 14 is a drawing illustrating a cross-spectrum between gamma interferon and gamma interferon wherein the amino acids in the region other than the active site region (132 to 162) are replaced by leucine.

FIG. 15 is a drawing illustrating a mixed frequency spectrum of gamma interferon.

FIG. 16 is a drawing illustrating a mixed frequency spectrum of gamma interferon receptor.

FIG. 17 is a drawing illustrating a mixed frequency spectrum of Gal4p.

FIG. 18 is a drawing illustrating a mixed frequency spectrum of an active site region (14 to 57) of Gal4p.

FIG. 19 is a drawing illustrating a mixed frequency spectrum of Gal7 promoter.

FIG. 20 is a drawing illustrating a mixed frequency spectrum of urokinase.

FIG. 21 is a drawing illustrating a mixed frequency spectrum of subtilisin.

FIG. 22 is a drawing illustrating a self-cross-spectrum of a prion protein.

FIG. 23 is a drawing illustrating a cross-spectrum between a prion protein and the 109-131 region of the prion protein.

FIG. 24 is a drawing illustrating a cross-spectrum between a prion protein and the prion protein wherein all the amino acid residues in the 109-131 region are replaced by leucine.

FIG. 25 is a drawing illustrating a mixed frequency spectrum of the 109-131 region of the prion protein.

FIG. 26 is a drawing illustrating a mixed frequency spectrum of the 110-126 region of the prion protein.

FIG. 27 is a drawing illustrating a self-cross-spectrum of an amyloid protein precursor.

FIG. 28 is a drawing illustrating a cross-spectrum between an amyloid protein precursor and the 650-680 region thereof.

FIG. 29 is a drawing illustrating a cross-spectrum between an amyloid protein precursor and the amyloid protein precursor wherein all the amino acid residues in the 650-680 region thereof are replaced by leucine.

FIG. 30 is a drawing illustrating a cross-spectrum between an amyloid protein precursor and the 289-364 region thereof.

FIG. 31 is a drawing illustrating a self-cross-spectrum of human growth hormone.

FIG. 32 is a drawing illustrating a cross-spectrum between human growth hormone and the human growth hormone wherein all the amino acid residues in the 109-217 region are replaced by leucine.

FIG. 33 is a drawing illustrating a cross-spectrum between human growth hormone and the human growth hormone wherein all the amino acid residues in the region other than the 109-217 region are replaced by leucine.

FIG. 34 is a drawing illustrating a cross-spectrum between human growth hormone and a salmon calcitonin precursor.

FIG. 35 is a drawing illustrating a cross-spectrum between human growth hormone and salmon calcitonin.

FIG. 36 is a drawing illustrating a mixed frequency spectrum of Ebola virus.

### DETAILED DESCRIPTION OF THE PREFERRED EMBODIMENTS

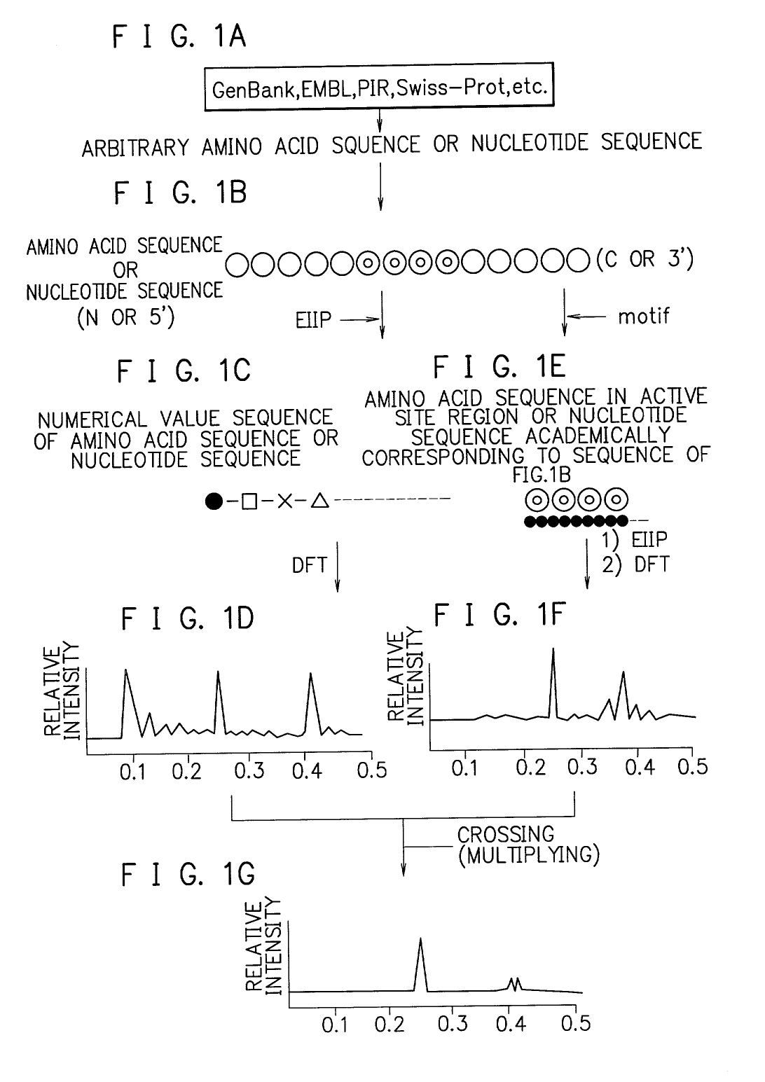

As a result of extensive studies, the present inventor has found a method for predicting biological•functional activity derived from an active site of a single protein or nucleotide sequence by comparing a natural-type or non-natural-type amino acid sequence (or nucleotide sequence) and an active site region present in the sequence. This method uses databases such as GenBank, EMBL, PIR, and SWISS-PROT (a protein (amino acid sequence) or nucleotide sequence in FIG. 1B registered in FIG. 1A).

Namely, the first step comprises giving EIIP (Electron-ion interaction potential) index values to the amino acids (or nucleotides) of the total amino acid sequence of a natural-type or non-natural-type arbitrary protein according to the method of Veljkovic et al. (V. Veljkovic et al., Cancer Biochem. Biophys., 9, 139-148 (1987); I. Cosic, IEEE, 41, 1101-1114 (1994)), subjecting the resulting numerical value sequence (hereinafter, referred to as “EIIP sequence”) in FIG. 1C to discrete Fourier transformation ((hereinafter, referred to as “DFT”), self-crossing the resulting frequency spectrum (hereinafter, referred to as “total amino acid sequence frequency spectrum) in FIG. 1D, and selecting non-characteristic frequency values derived from the whole protein molecule form a number of the peaks based on the relative intensity (height) of the peaks. In the selection of the peaks, 30 points of the peaks are selected in decreasing order of the frequency value. Preferably, a predetermined number of points within 3 to 12 are selected. The same method of selecting the peaks is also applied to in the following explanation.

The method of Veljkovic et al. will be specifically explained. That is, an EIIP sequence Fₙ (n=0, 1, 2, 3, . . . , L−3, L−2, L−1) (FIG. 1C) is prepared by giving EIIP index values (V. Veljkovic et al., Cancer Biochem. Biophys., 9, 139-148 (1987)) to the residues of an amino acid sequence having a chain length of L, a₀a₁a₂ . . . a_{L-3}a_{L-2}a_{L-1}. But, the amino acid sequence is extended so that the EIIP sequence becomes a power of 2. As the values of the numerical value sequence at extended part, an average EIIP index value of the amino acid sequence is adopted.

The EIIP index values to be given to amino acid residues are as follows: Leu 0.0000, Ile 0.0000, Asn 0.0036, Gly 0.0050, Val 0.0057, Glu 0.0058, Pro 0.0198, His 0.0242, Lys 0.0371, Ala 0.0373, Tyr 0.0516, Trp 0.0548, Gln 0.0761, Met 0.0823, Ser 0.0829, Cys 0.0829, Thr 0.0941, Phe 0.0946, Arg 0.0959, Asp 0.1263.

The resulting EIIP sequence Fₙ (n=0, 1, 2, 3, . L−3, L−2, L−1) (FIG. 1C) is treated according to the following equation of discrete Fourier transformation (DFT), i.e.,

Fₘ=Σ_{n=0, 1, 2, 3, . . . , L-3, L-2, L-1} fₙ exp(2mnπi/L)

to obtain a frequency spectrum Fₘ (FIG. 1D) . Fₘ satisfies a condition of periodicity. That is, F_{M-m}=Fₘ*. From the condition, only the Fₘ where m=0, 1, 2, . . . , L/2 has information.

The second step comprises giving EIIP index values to the amino acids (or nucleotides) of an active site region (FIG. 1E) comprising 2 to 64 amino acid residues containing any one or more of GT, AS, GA, ID, TR, SR, LK, TXW, VXH, MXH, WXP, AXC, GXS (wherein G, T. A, S, I, D, R, L, K, W, V, H, M, P, C, and X mean glycine, threonine, alanine, serine, isoleucine, aspartic acid, arginine, leucine, lysine, tryptophan, valine, histidine, methionine, proline, cysteine, and any of 20 kinds of amino acids, respectively) as a known motif pertinent to an active site in total amino acid sequence of an arbitrary protein, subjecting the EIIP sequence to DFT in a similar manner to the method in the first step, crossing the resulting frequency spectrum (hereinafter, referred to as “active site frequency spectrum”) in FIG. 1F and the total amino acid frequency in FIG. 1D obtained by subjecting the EIIP sequence of the total amino acid sequence to DFT, and selecting frequency values derived from an active site from a number of the peaks based on the relative intensity (height) of the peaks of the cross-spectrum (FIG. 1G).

The third step comprises giving EIIP index values to the amino acid residues of the total amino acid sequence (hereinafter, referred to as “replaced total amino acid sequence”) wherein all the amino acid residues of the above active site region comprising 2 to 64 amino acid residues are replaced by any one of 20 kinds of amino acids or all the amino acid residues of the region other than the active site region comprising 2 to 64 amino acid residues are replaced by any one of 20 kinds of amino acids without changing the active site region, determining a replaced total amino acid sequence frequency spectrum by subjecting the resulting EIIP sequence to DFT, and crossing the spectrum and the original total amino acid sequence frequency spectrum. And, from the result of the third step, the characteristic frequency values derived from the active site selected according to the methods of the first step and the second step are confirmed.

Using the above three-step method, a method for predicting biological•functional activity of an arbitrary protein by selecting efficiently characteristic frequency values derived from an active site of the protein has been found out, and thus the present invention has been accomplished.

The present inventor will exemplify a magainin precursor containing magainin 2 comprising 23 amino acid residues and having antibacterial activity. The magainin precursor comprises 300 amino acid residues which encodes five magainin 2 and one magainin 1 (M. Zasloff, Proc. Natl. Acad. Sci., U.S.A., 84, 5449-5453 (1987)). These amino acid sequences are registered in SWISS-PROT, and the nucleotide sequences in GenBank.

As a working hypothesis for developing a means for solving the problems, the present inventor has assumed that the amino acid sequence of magainin 2 (or magainin 1) is an active site region present in the amino acid sequence of the precursor.

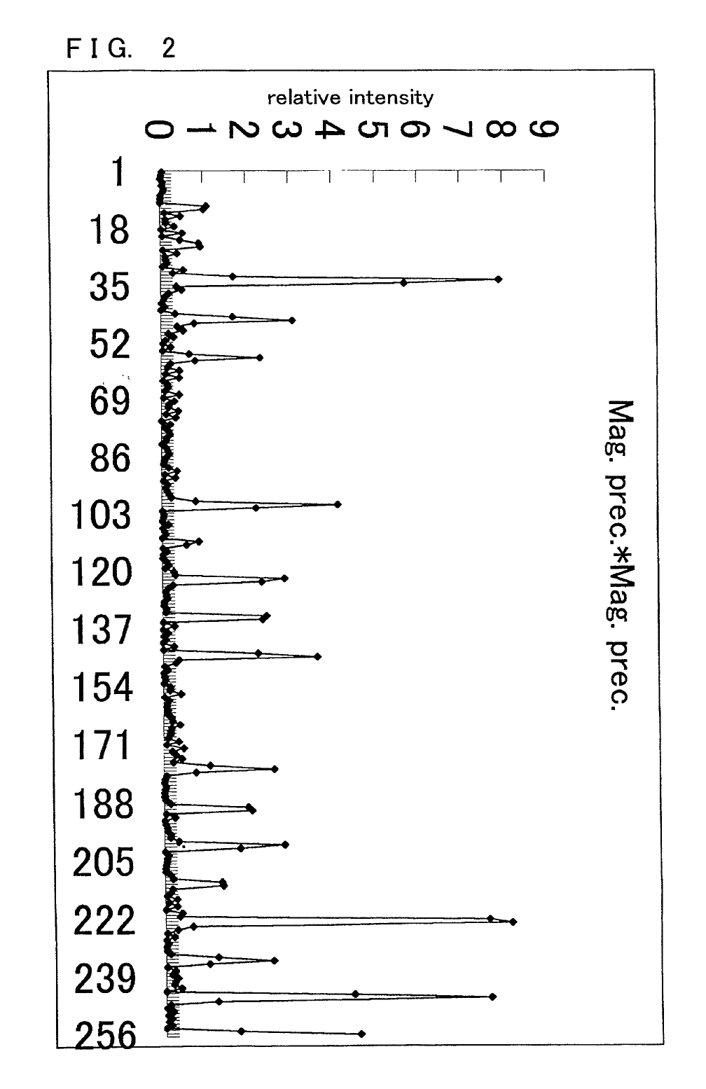

First, as the first step, the total amino acid sequence frequency spectrum of the magainin precursor (FIG. 2) is determined by self-crossing the total amino acid sequence frequency spectrum of the precursor according to the method of Veljkovic et al. (V. Veljkovic et al., IEEE 32, 337-341 (1985); V. Veljkovic et al., Cancer Biochem. Biophys., 9, 139-148 (1987)). From FIG. 2, the top 5 peaks are selected among 30 points for the sake of convenience based on the relative intensity of S/N (S means a signal peak and N means a noise peak) of the peaks (Table 1). The value in parenthesis shows the total number of points employed for the operation of DFT (the same is applied to the following).

TABLE 1
Frequency values derived from total sequence (512)
0.4355 0.0645 0.4785 0.4336 0.0664

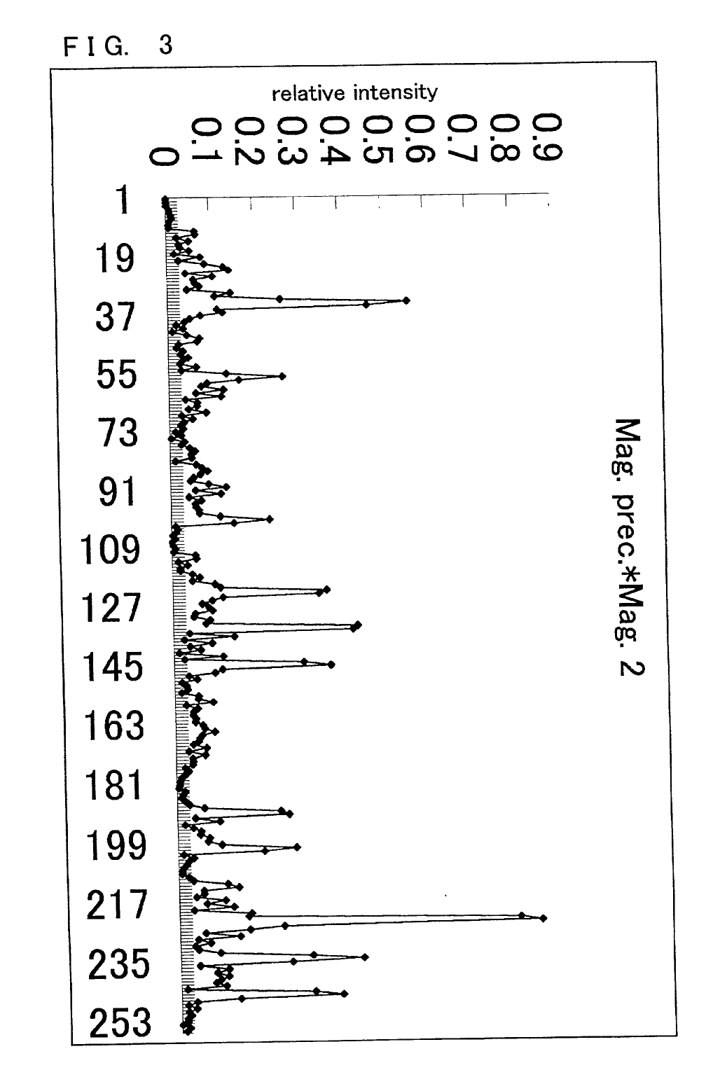

In the second step, for selecting peaks derived from the active site (magainin 2) from the 5 peaks, a total amino acid sequence frequency spectrum of the precursor and an active site frequency spectrum from the amino acid sequence of magainin 2 assumed as an active site region are determined and they are crossed (FIG. 3). The peaks derived from the active site obtained from FIG. 3 are shown in Table 2.

TABLE 2
Frequency values derived from active site (512)
0.4355 0.4336 0.0645 0.0664 0.2598

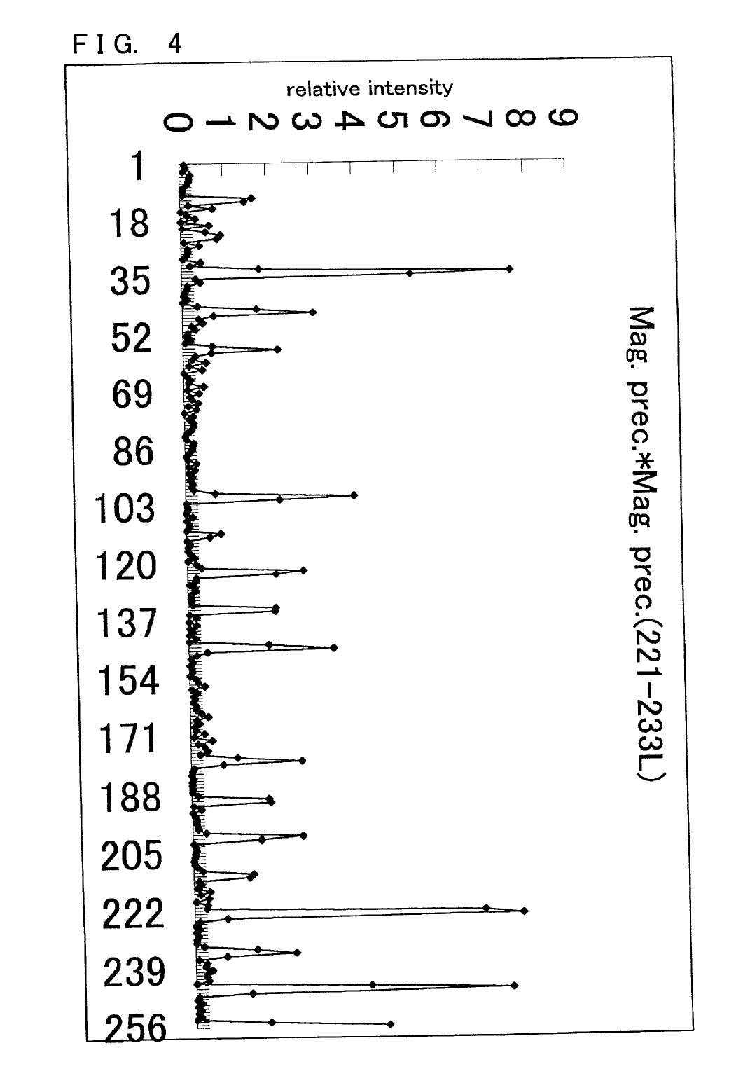

In the third step, in order to confirm whether the 5 peaks obtained in the second step are derived from the active site or not, the total amino acid sequence frequency spectrum and a replaced total amino acid sequence frequency spectrum wherein all the amino acid residues of the active site region and the precursor were replaced by leucine were crossed to obtain a total amino acid sequence frequency spectrum of the precursor as shown in FIG. 4. The selection of peaks under the same conditions as the cases of FIG. 2 and 3 affords Table 3. However, in this case, the amino acid residue for use in the replacement may also be any of 19 kinds of amino acids other than leucine.

TABLE 3
Frequency values of replaced total sequence (512)
0.4355 0.0645 0.4785 0.4336 0.0664

The tables 1, 2, and 3 are summarized to afford

TABLE 4
Magainin
Frequency values derived from total sequence (512)
0.4355 0.0645 0.4785 0.4336 0.0664
Frequency values derived from active site (512)
0.4355 0.4336 0.0645 0.0664 0.2598
Frequency values of replaced total sequence (512)
0.4355 0.0645 0.4785 0.4336 0.0664

As is understood form Table 4, the peaks at 0.0664, 0.2598, and 0.4336 are prominent peaks derived from the active site.

Furthermore, as a result of extensive studies, the present inventor has found an alternative method for predicting biological•functional activity or binding activity of an arbitrary protein which comprises determining a mixed frequency spectrum (FIG. 1G) (hereinafter, referred to as “mixed frequency spectrum”) by crossing a total amino acid sequence frequency spectrum (FIG. 1D) and a total nucleotide sequence frequency spectrum (FIG. 1F) obtained from an arbitrary natural-type or non-natural-type amino acid sequence (FIG. 1B) and a nucleotide sequence academically corresponding to the amino acid sequence (FIG. 1E) , respectively, through DFT, and selecting characteristic frequency values derived from an active site of the protein efficiently.

Namely, the first step comprises giving EIIP index values to the amino acids (or nucleotides) of an arbitrary total amino acid sequence according to the method of Veljkovic et al. (V. Veljkovic et al., Cancer Biochem. Biophys., 9, 139-148 (1987); I. Cosic, IEEE, 41, 1101-1114 (1994)), and subjecting the resulting values to DFT to prepare a total amino acid sequence frequency spectrum (FIG. 1D).

The EIIP index values to be given to amino acid residues are as follows:

Leu 0.0000, Ile 0.0000, Asn 0.0036, Gly 0.0050, Val 0.0057, Glu 0.0058, Pro 0.0198, His 0.0242, Lys 0.0371, Ala 0.0373, Tyr 0.0516, Trp 0.0548, Gln 0.0761, Met 0.0823, Ser 0.0829, Cys 0.0829, Thr 0.0941, Phe 0.0946, Arg 0.0959, Asp 0.1263.

The second step comprises giving EIIP index values to a nucleotide sequence academically corresponding to the amino acid sequence employed in the first step, and subjecting the EIIP sequence to DFT in a similar manner to the method in the first step to obtain a total nucleotide sequence frequency spectrum (FIG. 1F). The EIIP index values to be given to nucleotide residues are as follows: guanine: G 0.0806, adenine: A 0.1260, thymine (uracil: T(U) 0.1335 (0.0562)), cytosine: C 0.1340.

The third step comprises determining a mixed frequency spectrum (FIG. 1G) by crossing the total amino acid sequence frequency spectrum and the total nucleotide sequence frequency spectrum obtained in the first and second steps, and selecting frequency values derived from the active site.

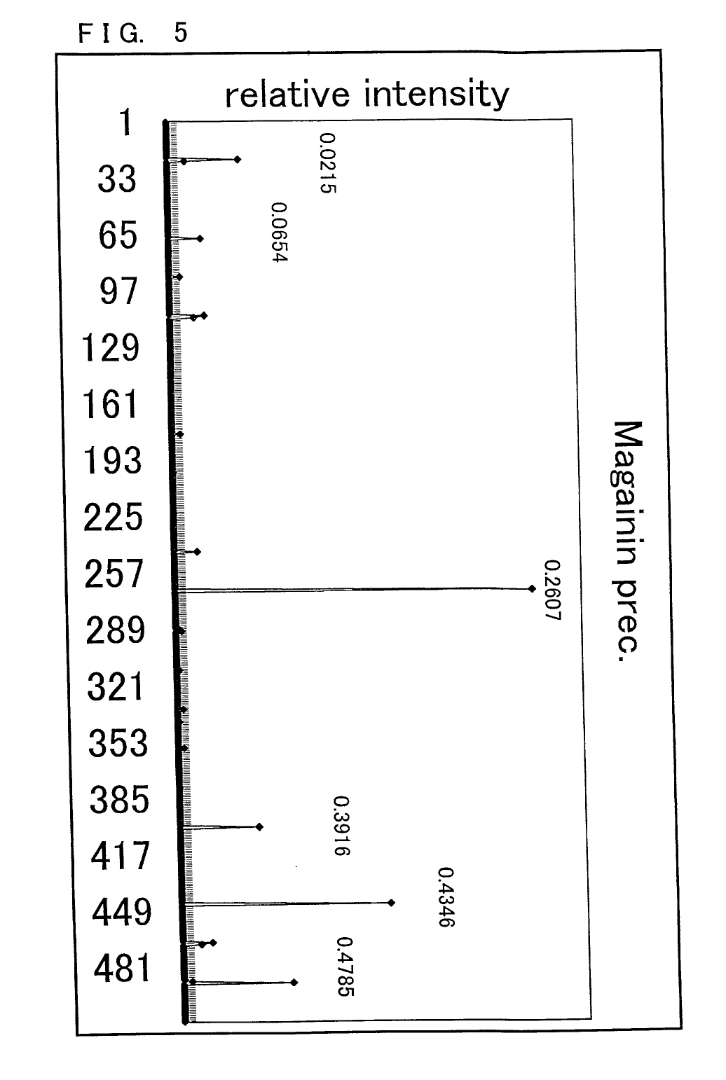

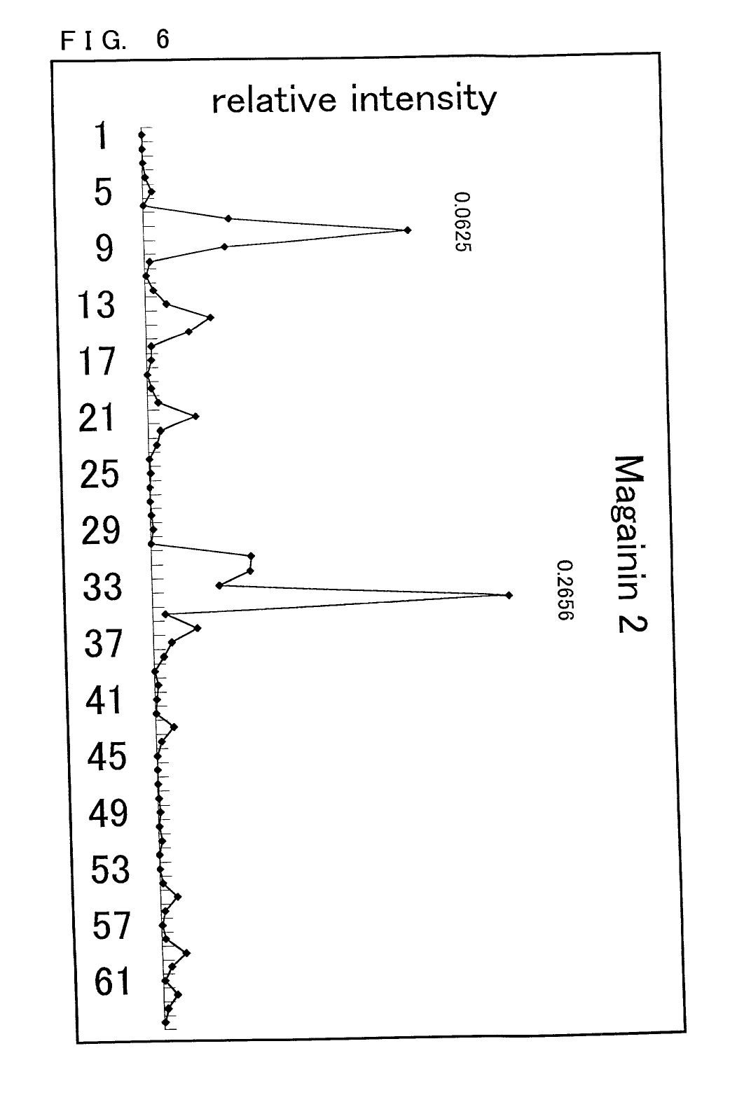

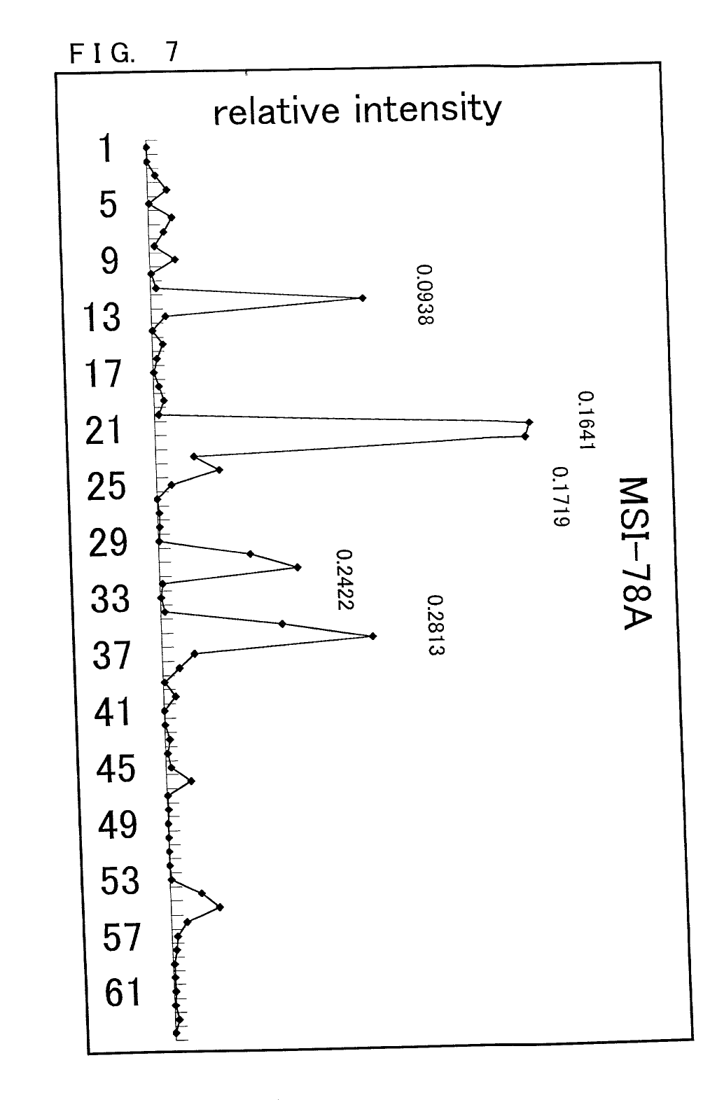

Namely, a mixed frequency spectrum (FIG. 5) is obtained from the amino acid sequence of a magainin 2 precursor and the nucleotide sequence thereof. From FIG. 5, prominent frequency values of 0.2607, 0.4346, 0.4785, 0.3916, 0.0215, 0.0654, and so forth are selected based on the relative intensity of the peaks. Similarly, the determination of a mixed frequency spectrum of magainin 2 assumed as an active site region affords FIG. 6. From FIG. 6, prominent frequency values of 0.2656, 0.0625, 0.2422, 0.2500, 0.0547, and so forth are selected based on the relative intensity of the peaks. From the comparison of the spectra of FIGS. 5 and 6, it is understood that 0.2607 and 0.0654 are the peaks derived from magainin 2 region among the above 6 frequency values of 0.0743 and so forth. And, these values are close to the values of 0.2598 and 0.0664 in Table 4. The present inventor has already reported decarboxylation activity of a magainin 2 derivative (MSI-78A) toward an oxaloacetate (N. Numao et al., Biol. Pharm. Bull., 22, 73-76 (1999)). However, magainin 2 hardly exhibits the decarboxylation activity. An amino acid sequence of MSI-78A and a nucleotide sequence academically corresponding thereto are assumed and a mixed frequency spectrum (FIG. 7) is determined. From FIG. 7, prominent frequency values are found to be 0.1641, 0.1719, 0.0938, 0.2813, and 0.2422 based on the relative intensity of the peaks. Therefore, the decarboxylation activity of MSI-78A toward an oxaloacetate is considered to be derived from 0.0938, 0.1641, 0.1719, and 0.2813.

On salmon calcitonin (I) known as a therapeutic agent for hyperclacemia and comprising 32 amino acids and a precursor thereof (136 amino acids), as a reference example wherein only one active site region exists, the present inventor has selected characteristic frequency values derived from the active site. The amino acid sequence is registered in SWISS-PROT.

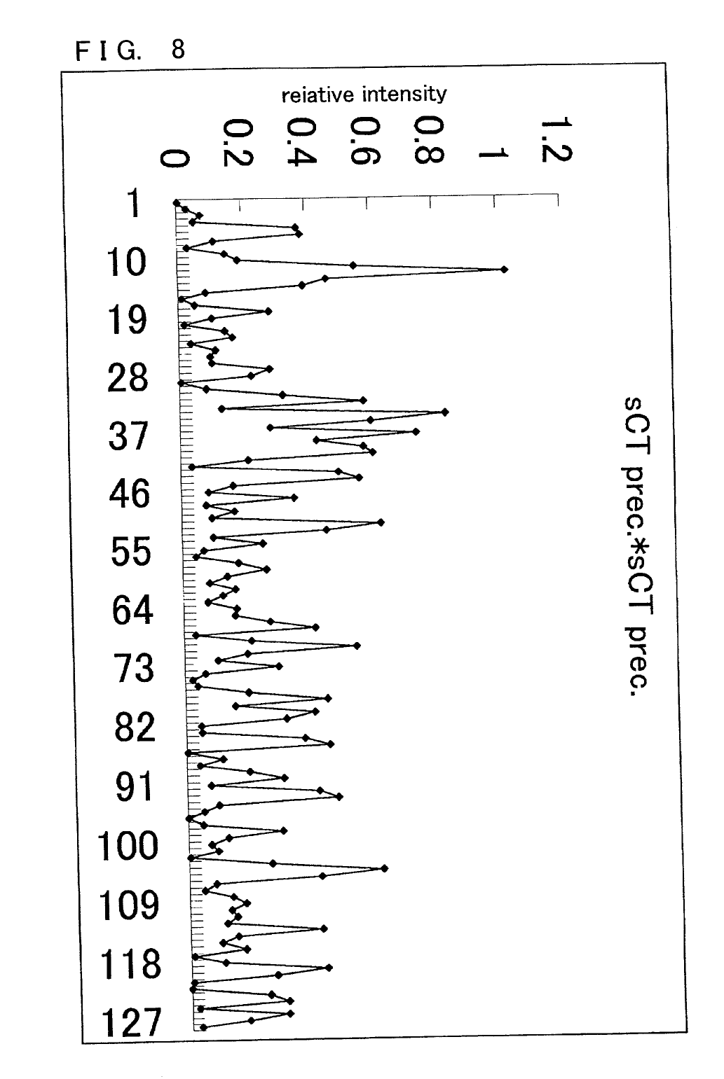

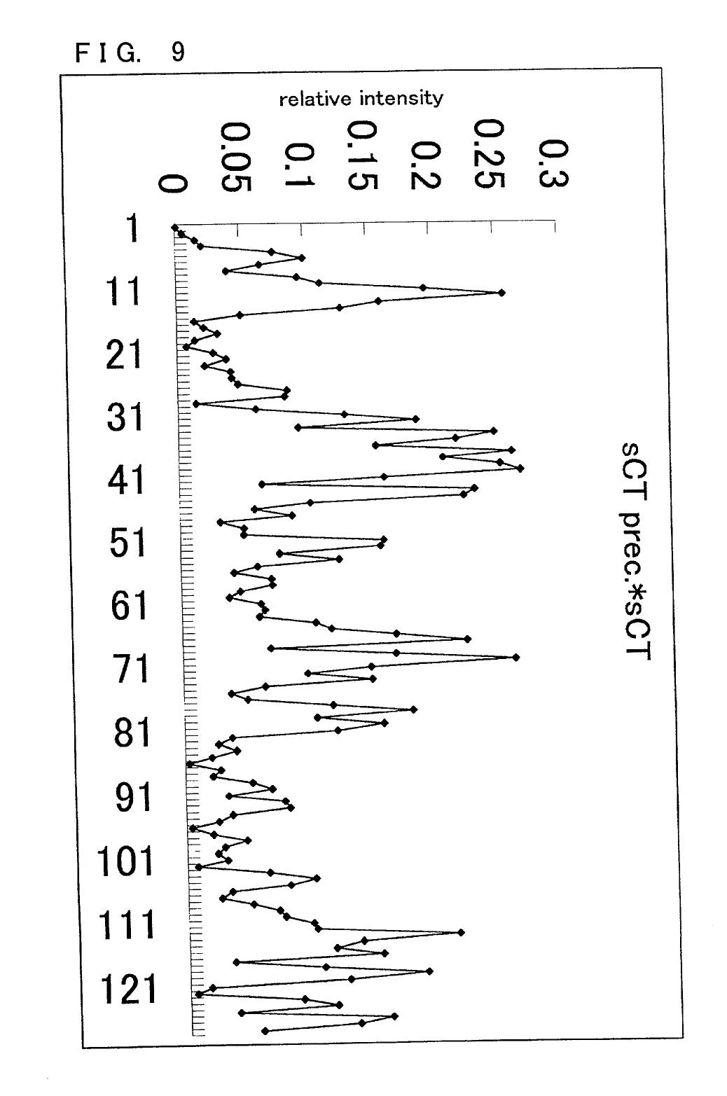

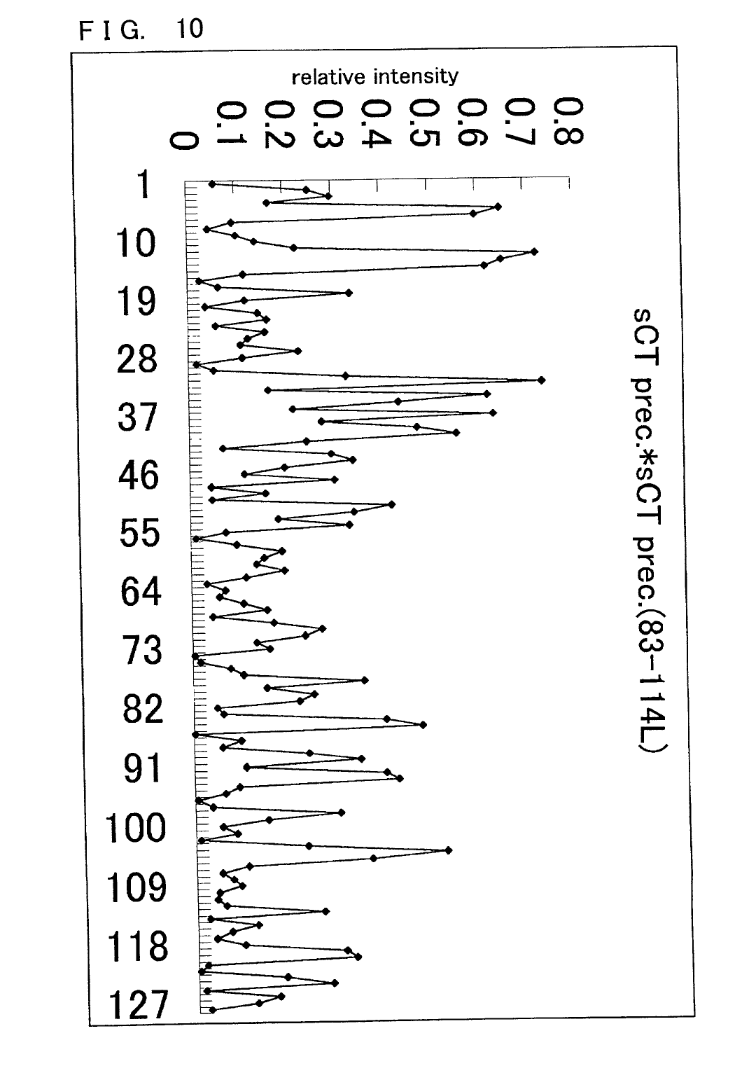

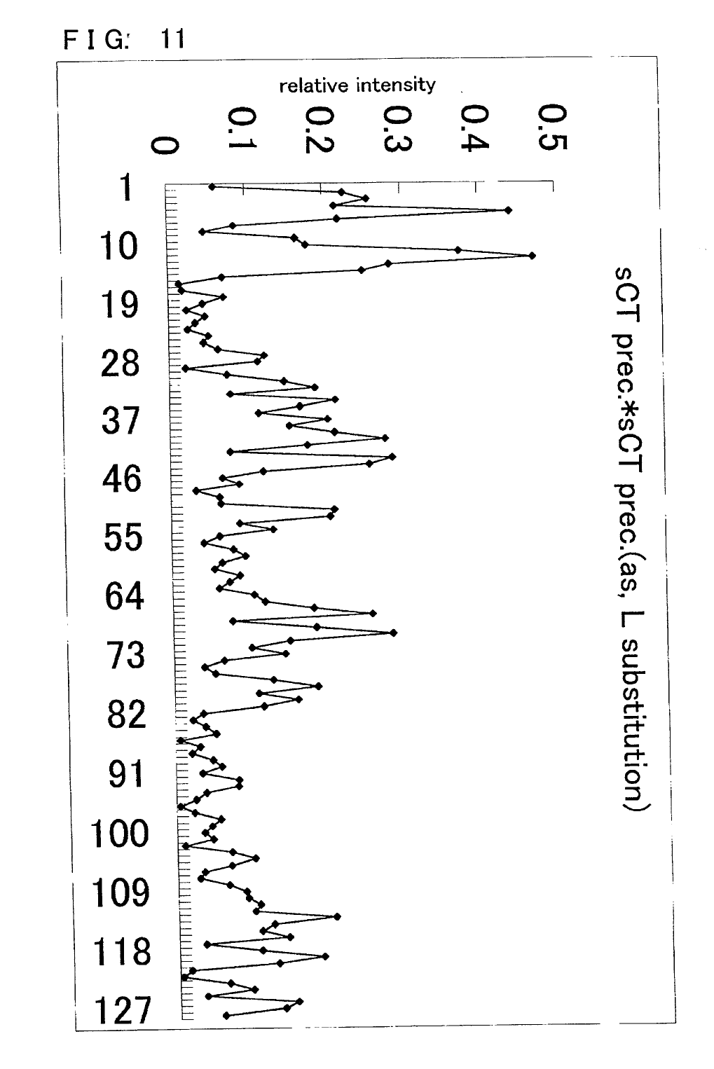

As the first step, the total amino acid sequence frequency spectrum of the salmon calcitonin (I) precursor (FIG. 8) is determined by self-crossing the total amino acid sequence of the precursor, and 5 peaks derived from the whole precursor molecule are selected based on the relative intensity of the peaks. In the second step, an active site frequency spectrum of salmon calcitonin (I) assumed as an active site region and the total amino acid sequence frequency spectrum of the salmon calcitonin precursor are crossed, and from the cross-spectrum (FIG. 9), the peaks derived from salmon calcitonin (I) are selected. In the third step, in order to confirm whether the 5 peaks obtained in the second step are derived from the salmon calcitonin site or not, a replaced total amino acid sequence frequency spectrum wherein the salmon calcitonin region (amino acid number of 84 to 114) present in the precursor are replaced by leucine and the total amino acid sequence frequency spectrum are crossed and from the resulting cross-spectrum (FIG. 10), frequency values I of the replaced total sequence are determined. The selection of peaks under the same conditions as the cases of magainin and summarization thereof afford Table 5. Moreover, as an alternative of the third step, a replaced total amino acid sequence frequency spectrum wherein all the amino acid residues other than the amino acid sequence of the region (amino acid number of 84 to 114) encoding the salmon calcitonin in the amino acid sequence of the precursor were replaced by leucine is operated according to the above method (FIG. 11). The result is described in Table 5 as frequency values II of the replaced total amino acid sequence. However, in this case, the amino acid residue for use in the replacement may also be any of 19 kinds of amino acids other than leucine.

TABLE 5
Salmon calcitonin
Frequency values derived from total sequence (256)
0.0469 0.1328 0.1445 0.1992 0.4063
Frequency values derived from active site (256)
0.1563 0.2734 0.1445 0.0469 0.1523
Frequency values I of replaced total sequence (256)
0.1250 0.0469 0.0508 0.0195 0.1445
Frequency values II of replaced total sequence (256)
0.0469 0.0195 0.1680 0.0508 0.2734

From Table 5, the peaks at 0.1445, 0.1523, 0.1563 and 0.2734 are peaks derived from salmon calcitonin (I). Since the protein showing these values are not described in the known literatures (V. Veljkovic et al., Cancer Biochem. Biophys., 9, 139-148 (1987); I. Cosic, IEEE, 41, 1101-1114 (1994)), novel biological•functional activity of salmon calcitonin (I) is unclear. However, biological•functional activity derived from at least two frequency values may be expected in salmon calcitonin (I).

Furthermore, on gamma interferon (R. Wetzel et al., Protein Eng., 3, 611-623 (1990)) wherein the protein whose C-terminal region of the amino acid sequence is deleted is known to exhibit almost no antivirus activity, the present inventor has select characteristic frequency values derived from the active site. The amino acid sequence is registered in SWISS-PROT and comprises 166 amino acid residues. The active site is known to be present at 151-154 residues in the amino acid sequence of gamma interferon. However, in order to clarify the object of the present invention, the active site (247±15) predicted by the present inventor using 13 kinds of motifs is adopted (N. Numao et al., Biol. Pharm. Bull., 16, 1160-1163 (1993)).

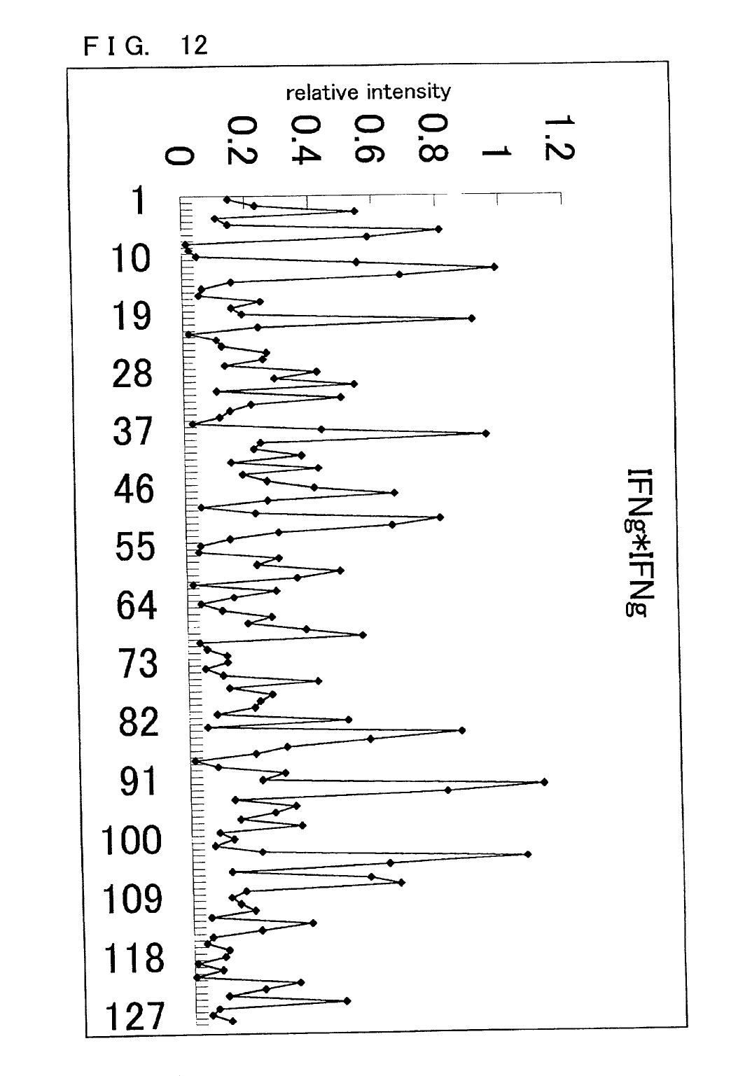

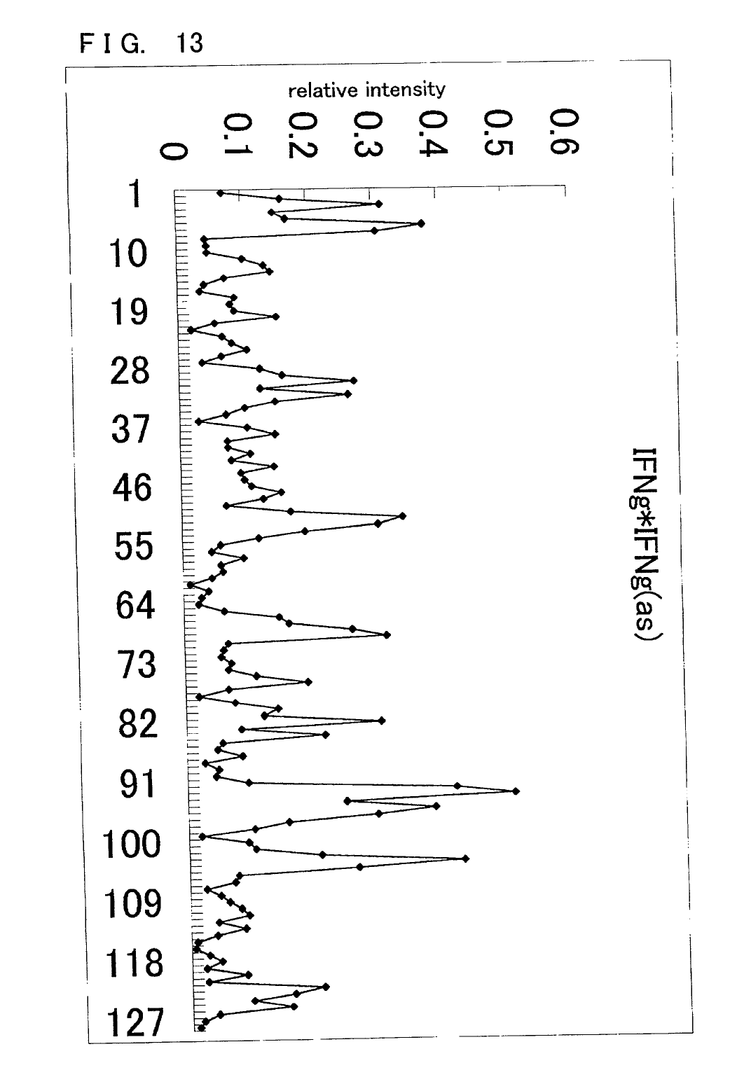

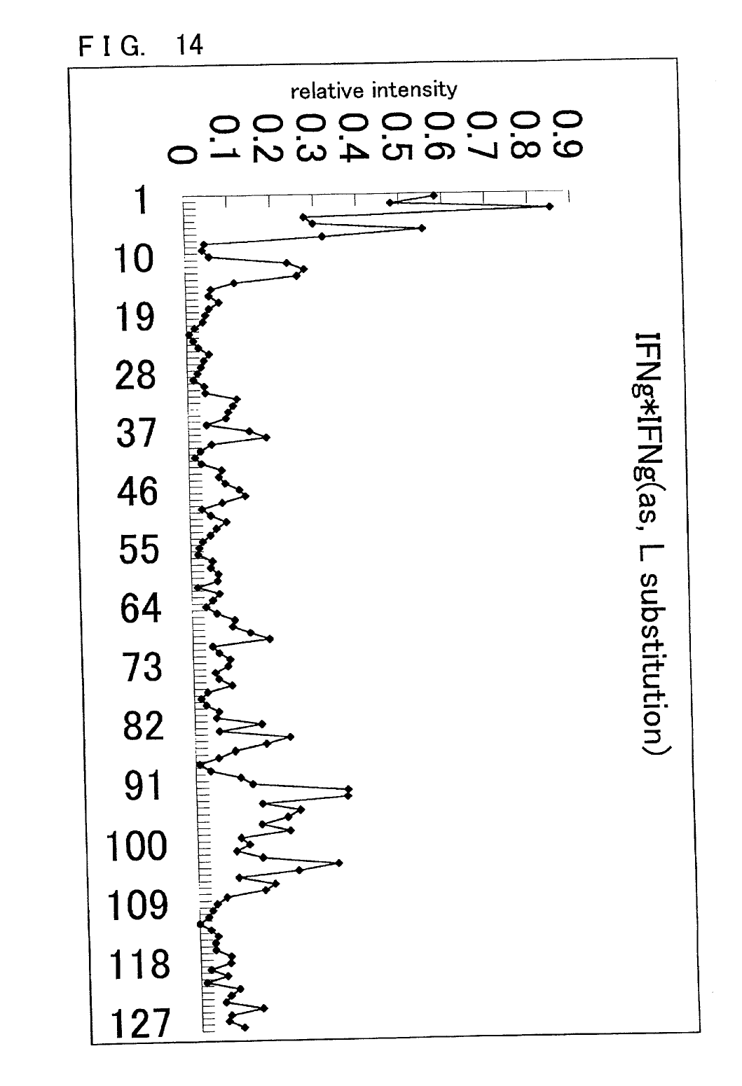

As the first step, a total amino acid sequence frequency spectrum of gamma interferon (FIG. 12) is determined by self-crossing the total amino acid sequence of gamma interferon comprising 166 amino acid residues. Top 5 peaks are selected based on the relative intensity of the peaks. In the second step, an active site frequency spectrum of the region of 132 to 162 which is the active site of gamma interferon and the total amino acid sequence frequency spectrum of the total amino acid sequence of gamma interferon are crossed, and top 5 peaks derived from the region 132 to 162 are selected based on the relative intensity of the peaks (FIG. 13). In the third step, in order to confirm whether the 5 peaks obtained in the second step are derived from the region of 132 to 162 or not, a replaced total amino acid sequence frequency spectrum wherein the active site region (132 to 162) present in gamma interferon are replaced by leucine and the total amino acid sequence frequency spectrum are crossed to obtain a total amino acid sequence frequency spectrum of gamma interferon as shown in FIG. 14. Moreover, as an alternative of the third step, a replaced total amino acid sequence frequency spectrum wherein all the amino acid residues other than the amino acid sequence of 132 to 162 in the amino acid sequence of gamma interferon are replaced by leucine is operated according to the usual method. The result is described in Table 6 as frequency values II of the replaced total amino acid sequence (the values of 0 to 0.015 are ignored because they are apparently not derived from the active site). However, in this case, the amino acid residue for use in the replacement may also be any of 19 kinds of amino acids other than leucine.

TABLE 6
Gamma interferon
Frequency values derived from total sequence (256)
0.3594 0.4023 0.0469 0.1484 0.0781
Frequency values derived from active site (256)
0.0234 0.3594 0.3633 0.0273 0.4023
Frequency values I of replaced total sequence (256)
0.3594 0.0469 0.0781 0.1484 0.0117
Frequency values II of replaced total sequence (256)
0.0234 0.3594 0.3633 0.4023 0.0273

From Table 6, the peaks at 0.0234, 0.0273, and 0.3633 are peaks derived from predicted active site of gamma interferon. According to known literatures (V. Veljkovic et al., Cancer Biochem. Biophys., 9, 139-148 (1987); I. Cosic, IEEE, 41, 1101-1114 (1994)), the frequency value derived from the whole molecule of the interferon is 0.082±0.008. However, based on the prominence of the peaks, the frequency values derived from the active site of the present invention of 0.0117 to 0.0234 may be pertinent to the antivirus activity of gamma interferon. From the known literatures, the value of 0.0234 is coincident with the value of hemoglobin.

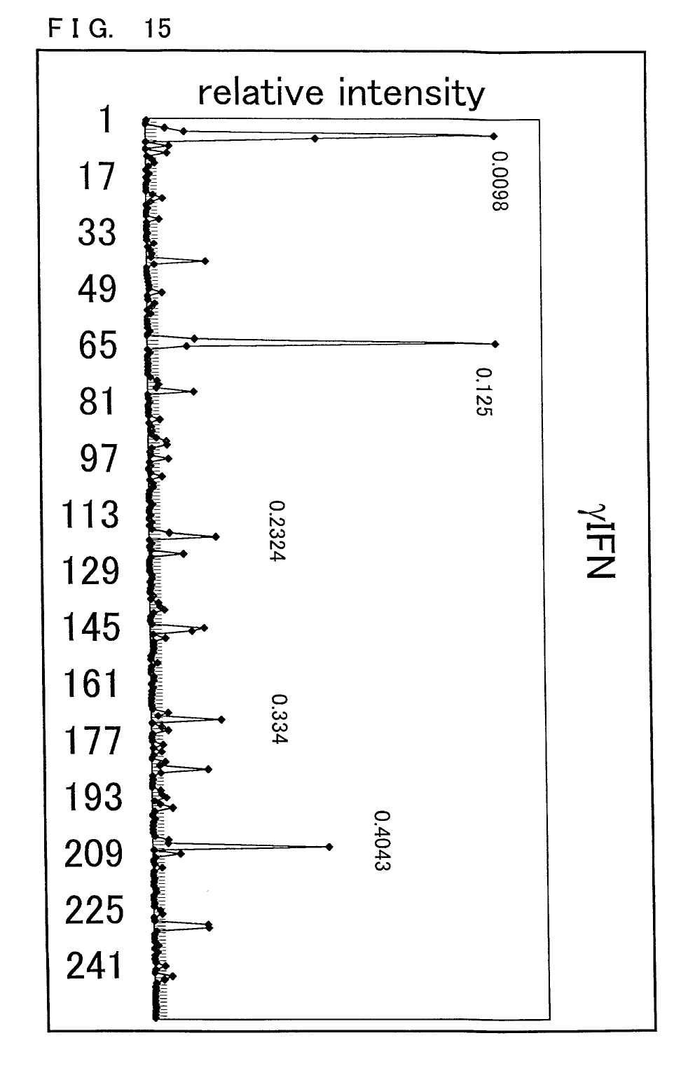

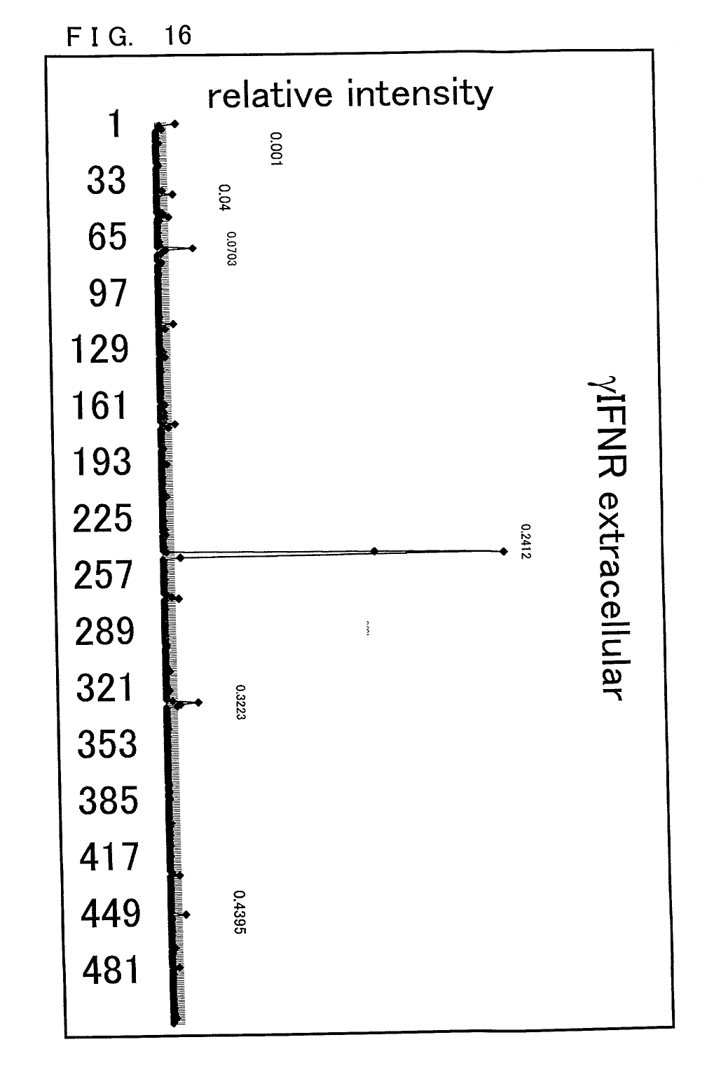

Furthermore, the present inventor describes an alternative method for clarifying the method of the present invention. Namely, total frequency spectra of the amino acid sequence of gamma interferon and a nucleotide sequence academically corresponding thereto are crossed to determine a mixed frequency spectrum (FIG. 15), and peaks derived from the active site of gamma interferon are selected. From FIG. 15, 0.0098, 0.1250, 0.4043, 0.0117, 0.334, 0.2324, and so forth were selected based on the relative intensity of the peaks. Among the values, 0.0010 is close to 0.0098 and 0.0117 derived from gamma interferon. Furthermore, as a result of extensive studies of the DFT analysis of the present method, certain regularity with regard to the bonding between frequency region of gamma interferon (m/L≦0.5) and frequency region of gamma interferon receptor (0.5-m/L) has been found out, and thus the present invention has been accomplished. Namely, a mixed frequency spectrum (FIG. 16) is determined from the amino acid sequence of extracellular region of gamma interferon receptor (M. Aguet et al., Cell 55, 273-280 (1988)) which is a receptor of gamma interferon and a nucleotide sequence academically corresponding to the amino acid sequence, and characteristic frequency values thereof are selected. From FIG. 16, based on the relative intensity of the peaks, 0.2412, 0.0703, 0.3223, 0.0010, 0.0400, 0.4395, and so forth in the frequency region (0.5-m/L) are selected as characteristic frequency values of the extracellular region. Among the values, 0.0010, 0.2412, and 0.3223 are close to the values (0.0098, 0.2324, and 0.3340) derived from the active site of gamma interferon. Examples wherein ligand/receptor bonding relationship can be explained according to a similar method include HIVgp120and CD4 receptor, Poliovirus coatprotein VP1 and poliovirus receptor, IL-2 and IL-2 receptor, TNF-α (or TNF-β) and 55kd TNF receptor, or Insulin and Insulin Receptor. Furthermore, the values of prominent peaks of gamma interferon receptor overlaps more frequently with the values of gamma interferon than the prominent values of IL-2, TNF-α, TNF-β, Insulin, or the like. Therefore, according to the method of the present invention, other protein selectively binding to an arbitrary protein can be searched for.

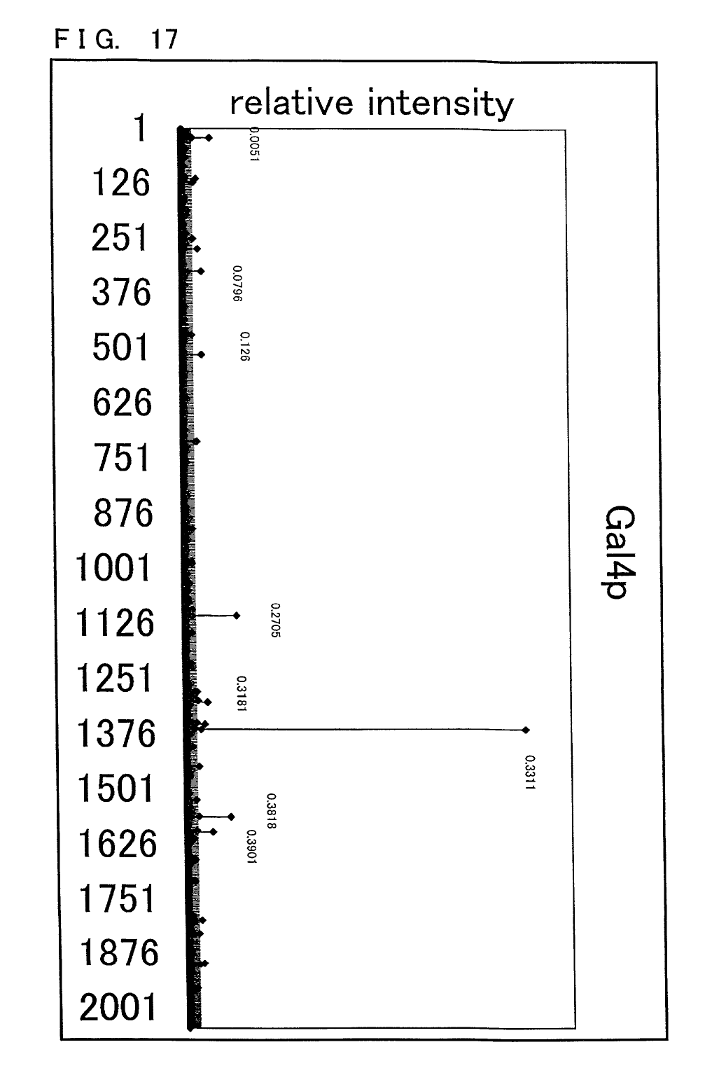

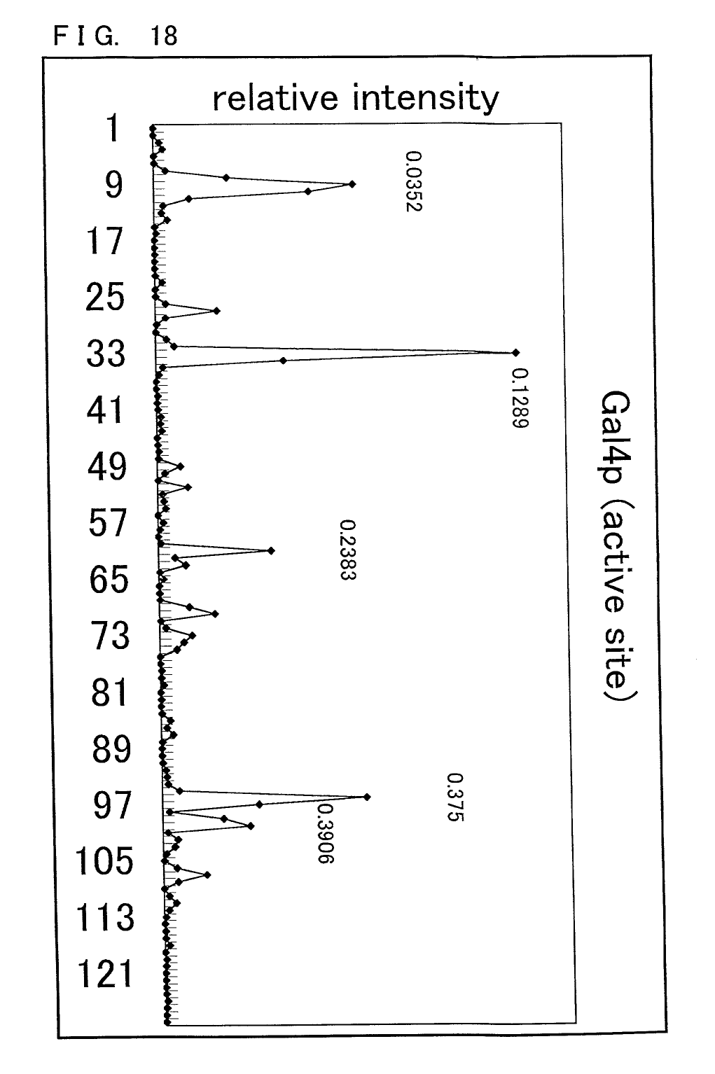

The present inventor has further selected characteristic frequency values derived from the active site of a yeast transcription factor protein Gal4p (A. S. Laughon et al., Mol. Cell Biol., 4, 260-267 (1984)) according to a novel method. It is reported that Gal4p protein is constituted by 881 amino acids and the DNA-protein binding domain exists the region of 14 to 57 (M. Johnston, Microbiol. Rev., 51, 458-476 (1987)). Namely, a mixed frequency spectrum (FIG. 17) is determined by crossing total frequency spectra obtained from the amino acid sequence of Gal4p and a nucleotide sequence academically corresponding to the amino acid sequence. From FIG. 17, based on the relative intensity of the peaks, 0.3311, 0.2705, 0.3818, 0.0051, 0.3901, 0.0796, 0.3181, 0.1280, and so forth are selected as prominent frequency values derived from Gal4p. Next, a mixed frequency spectrum (FIG. 18) is determined by crossing total frequency spectra obtained from the amino acid sequence of the region of 14 to 57 and a nucleotide sequence academically corresponding thereto. From FIG. 18, based on the relative intensity of the peaks, 0.1289, 0.3750, 0.0352, 0.0391, 0.1328, 0.2383, 0.3789, 0.3908, and so forth are selected as prominent frequency values derived from the region of 14 to 57. Therefore, form FIGS. 17 and 18, among the prominent frequency values derived form Gla4p, at least 0.1280 and 0.3818 are pertinent to DNA-protein binding. Accordingly, there is a possibility that the total amino acid sequence frequency spectrum of Gal4p contains frequency values derived from the active site.

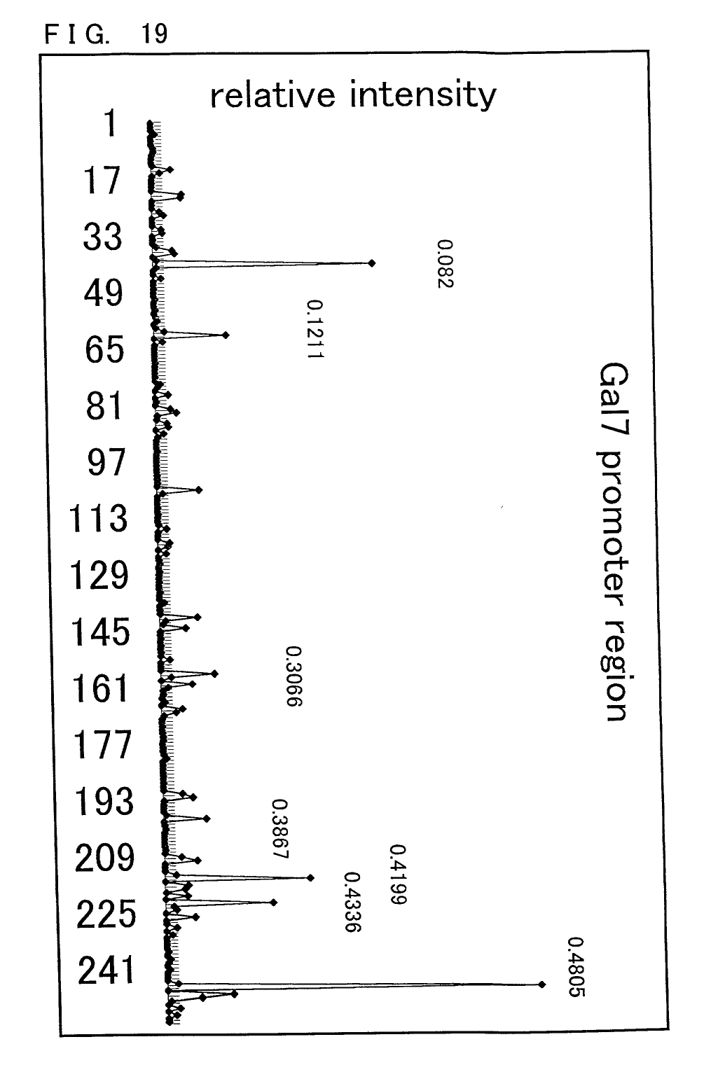

On the other hand, the present inventor has extensively studied and a homogeneous nucleotide sequence frequency spectrum (FIG. 1G) (hereinafter, referred to as “homogeneous nucleotide sequence frequency spectrum”) is determined by crossing total nucleotide sequence frequency spectra (FIG.s 1D and 1F) obtained by subjecting to DFT from an arbitrary nucleotide sequence (FIG. 1B) and a nucleotide sequence (FIG. 1E) which can be formed by hydrogen bonding to the single-strand nucleotide, and the resulting prominent characteristic frequency values are selected. The EIIP index values to be given to nucleotide residues are as follows: guanine: G 0.0806, adenine: A 0.1260, thymine (uracil: T(U) 0.1335 (0.0562)), cytosine: C 0.1340. Namely, a Gal7 promoter region is constituted by 350 nucleotides and binds to a yeast transcription factor protein Gal4p (A. S. Laughon et al., Mol. Cell Biol., 4, 260-267 (1984)) (R. J. Bram et al., EMBO J., 5, 603-608 (1986)). A nucleotide sequence frequency spectrum (FIG. 1D) of the single-strand nucleotide sequence is determined. Next, a nucleotide sequence frequency spectrum (FIG. 1F) is determined from a single-strand nucleotide sequence (FIG. 1E) which can be formed by hydrogen bonding to the above nucleotide sequence. Further, a homogeneous nucleotide sequence frequency spectrum (FIG. 1F) (FIG. 19) is determined by crossing these two nucleotide sequence frequency spectra. From FIG. 19, based on the relative intensity of the peaks, 0.4805, 0.0820, 0.4210, 0.4336, 0.1211, 0.4844, 0.3066, 0.2051, 0.3867, and so forth in frequency region (0.5-m/L) are selected as characteristic frequency values in the Gal promoter region. Among these values, 0.0820, 0.1211, 0.3066, and 0.3867 are almost coincident with 0.0796, 0.1280, 0.3181, and 0.3818 in the Gal4p case described above. Therefore, in consideration of the overlapping degree of prominent characteristic frequency values derived from Gal4p and the Gal7 promoter region, it is explainable that these two polymeric compounds can bind each other. Examples whose binding can be reasonably explained by such a method include a peptide derived from a prion protein and a synthetic RNA segment (S. Weiss et al., J. Virol., 71, 8790-8797 (1997)), OCT1 and TNF promoter region (J. C. Knight et al., Nature Genetics 22, 145-150 (1999)), pho4p (or pho2p) and Pho5 transcription region (Y. Ohshima, Genes Genet. Syst., 72, 323-334 (1997)), and the like. Therefore, according to the method of the present invention, it is also possible to predict a binding protein from a desired nucleotide sequence and/or a binding nucleotide sequence from a desired protein.

The present inventor has found out a method for predicting similarity of the biological•functional activity between two proteins, which comprises:

1) determining an arbitrary mixed frequency spectrum by crossing a total amino acid sequence frequency spectrum of the total amino acid sequence of a natural-type or non-natural-type arbitrary protein and a total nucleotide sequence frequency spectrum of a nucleotide sequence academically corresponding thereto, and selecting prominent characteristic frequency values thereof,

2) determining a mixed frequency spectrum by crossing a total amino acid sequence frequency spectrum of the total amino acid sequence of natural-type or non-natural-type another protein and a total nucleotide sequence frequency spectrum of a nucleotide sequence academically corresponding thereto, and selecting prominent characteristic frequency values thereof, and then measuring overlapping number of prominent characteristic frequency values selected based on the relative intensity of the peaks in the two mixed frequency spectra.

Namely, characteristic frequency values derived from active sites of urokinase (UK) classified as a serine protease (W. E. Holmes et al., Biotechnology 3, 923-929 (1985)) and subtilisin (J. A. Wells et al., Nucl. Acids Res., 11, 7911-7925 (1983)) are selected and similarity of biological•functional activity thereof is examined. The amino acid sequences are registered in SWIAA-PROT and the nucleotide sequence in GenBank.

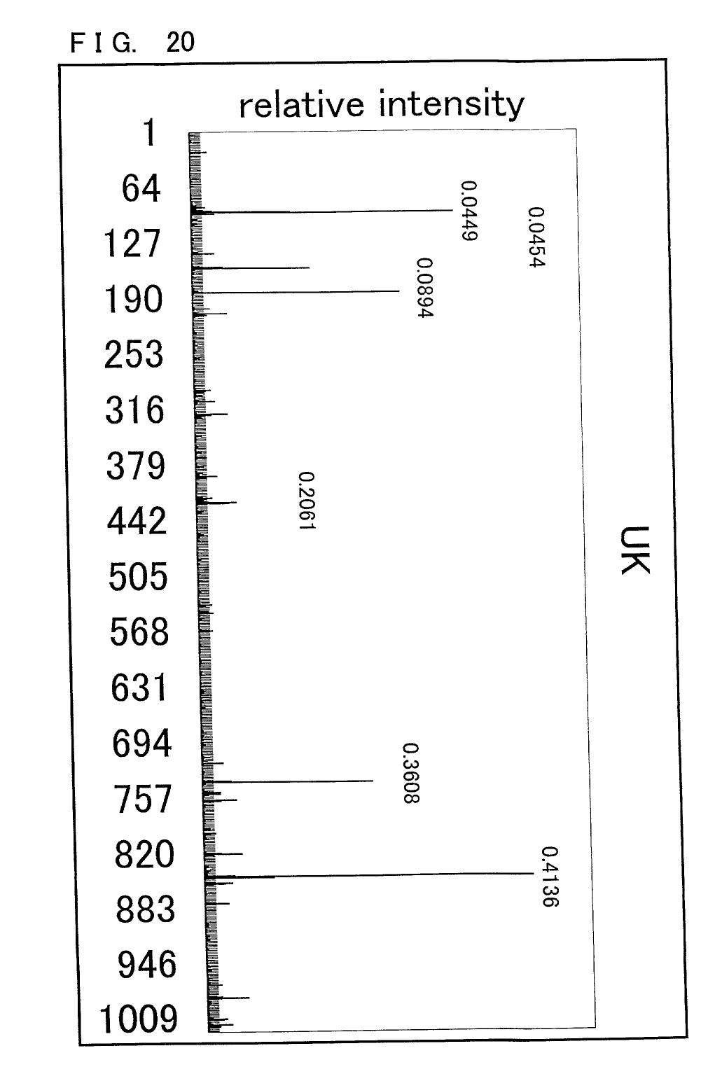

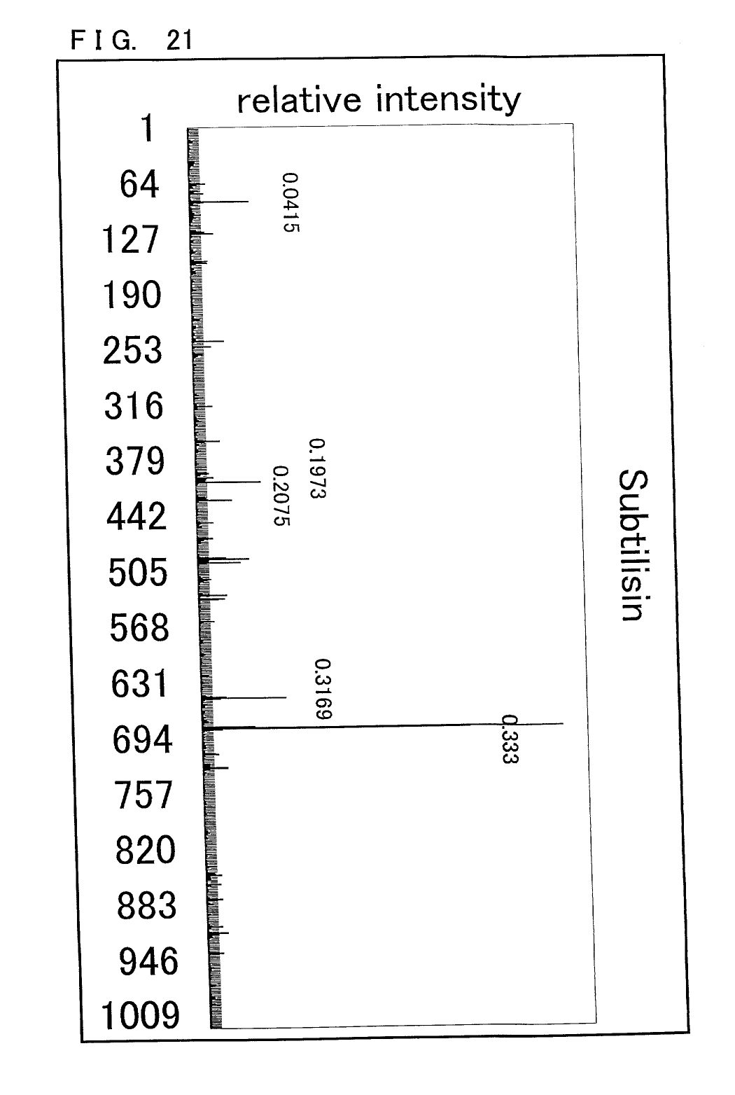

That is, as the first step, a total amino acid sequence frequency spectrum of the total amino acid sequence (431aa) of UK and a total nucleotide sequence frequency spectrum of a nucleotide sequence (1293na) academically corresponding thereto are determined. Then, a mixed frequency spectrum (FIG. 20) of UK is determined by crossing these spectra and, based on the relative intensity of the peaks, prominent characteristic frequency values thereof are selected. The prominent characteristic frequency values of UK are 0.4136, 0.0454, 0.0898, 0.3608, 0.0762, 0.0449, 0.4141, 0.4814, 0.2061, 0.4009, and so forth. As the second step, a total amino acid sequence frequency spectrum of the total amino acid sequence (376aa) of subtilisin and a total nucleotide sequence frequency spectrum of a nucleotide sequence (1128 na) academically corresponding thereto are determined. Then, a mixed frequency spectrum (FIG. 21) of subtilisin is determined by crossing these spectra and, based on the relative intensity of the peaks, prominent characteristic frequency values thereof are selected. The prominent characteristic frequency values of subtilisin are 0.3330, 0.3335, 0.3169, 0.1973, 0.0415, 0.3325, 0.2397, 0.2412, 0.2075, 0.1191, and so forth. From FIGS. 20 and 21, 3 or more of overlapping peaks (0.0454, 0.0449, 0.2061 and 0.0415, 0.1973, 0.2075) can be selected among the prominent characteristic frequency values. As typical examples wherein similarity of biological•functional activity can be explained, TNF-α and TNF-β, the above-described yeast transcription factors of pho4p and pho2p, and the like can be mentioned. Therefore, it is possible to predict a protein having biological•functional activity similar to that of desired protein according to the method of the present invention.

The present inventor has further disclosed that, when a homogeneous nucleotide sequence frequency spectrum is determined by crossing a total nucleotide sequence frequency spectrum of a nucleotide sequence of a promoter region and a total nucleotide sequence frequency spectrum of a nucleotide sequence academically corresponding thereto, and prominent characteristic frequency values thereof are selected based on the relative intensity of the peaks, the values contains prominent characteristic frequency values derived from a motif. Furthermore, as one example, the method can be applied to the interaction between exon and intron on a genome sequence. Such examples include interaction between exon and intron on non-mature mRNA such as CD4 receptor.

In summary, the present inventor has found out a method for predicting biological•functional activity (or binding activity) of an arbitrary amino acid sequence (or nucleotide sequence) by comparing:

1) a total amino acid sequence frequency spectrum obtained by giving EIIP (Electron-ion interaction potential) index values to the amino acid residues of an arbitrary amino acid sequence and subjecting the resulting EIIP sequence to DFT, and/or

2) an active site frequency spectrum obtained by giving EIIP index values to the amino acids of an amino acid sequence region, which is composed of 2 to 64 amino acid residues present in an arbitrary amino acid sequence and containing at least one known motif pertinent to an active site and subjecting the resulting EIIP sequence to DFT, and/or

3) a total nucleotide sequence frequency spectrum obtained by giving EIIP index values to the nucleotide residues of a nucleotide sequence region academically corresponding to an amino acid sequence, and subjecting the resulting EIIP sequence to DFT, and/or

4) a total nucleotide sequence frequency spectrum obtained by giving EIIP index values to the nucleotide residues of an arbitrary nucleotide sequence and subjecting the resulting EIIP sequence to DFT, and

5) a total nucleotide sequence frequency spectrum obtained by giving EIIP index values to the nucleotide residues of a nucleotide sequence which binds to an arbitrary nucleotide sequence through hydrogen bonding, and subjecting the resulting EIIP sequence to DFT; and

the arbitrary amino acid sequence or nucleotide sequence being one whose function is unknown and being originated in natural-type or non-natural-type and registered in databases such as GenBank, EMBL, PIR, and SWISS-PROT.

Accordingly, as is apparent from the process of the method of the present invention, all of classified lists, functional activity, and correlation charts of a natural-type or non-natural-type arbitrary protein (or nucleotide sequence) predicted by a program or a storage medium prepared based on the above concept using a mathematical procedure (Fourier analysis, wavelet analysis, or the like) are not limited to the examples of the present invention. Therefore, it is also apparent that, since it is easy to search for a novel protein or nucleotide sequence expectable to have a desired interaction with an arbitrary protein or nucleotide sequence from the lists prepared based on the above concept, such development of a program and a storage medium fall within the claims of the present invention so far as the basic concept of the present invention is employed. Furthermore, summarized and classified lists and/or functional activity, binding correlation charts of a natural-type or non-natural-type arbitrary protein (or nucleotide sequence) predicted by a program or a storage medium prepared based on the above concept using a mathematical procedure (Fourier analysis, wavelet analysis, or the like), employing constants or simple integers of hydrophilicity or hydrophobicity of the above amino acid residues and nucleotide residues as substitutes of the EIIP index values thereof.

Moreover, since an active site of an arbitrary sequence (amino acid sequence, nucleotide sequence) can be narrowed down based on the prominent frequency values obtained in the mixed frequency spectra of the magainin precursor, gamma interferon, and Gal4p, the method of the present invention is not limited to only the means for predicting from an active site region. In addition, such development of a program or a storage medium with regard to narrowing down the active site of an arbitrary amino acid sequence or nucleotide sequence also fall within the claims of the present invention.

Furthermore, it has already been found that the results obtained in the present method are the same even when the direction of the amino acid sequence is changed from N->C to C->N or the direction of the nucleotide sequence from 5′->3′to 3′->5′, so that the direction of these sequences is not restricted.

Moreover, since the method of the present invention is based on investigation of fundamental principle or concept with regard to an active site present in an amino acid sequence or nucleotide sequence, the claim regarding the application of predicted functional activity of a protein or nucleotide sequence relates to not only a pesticide, medicament, or the like as a therapeutic agent, but also prevention or diagnosis of a hereditary disease, a pestilence, or the like.

### EXAMPLE1

Prediction of Biological•Functional Activity of Normal Prion Protein

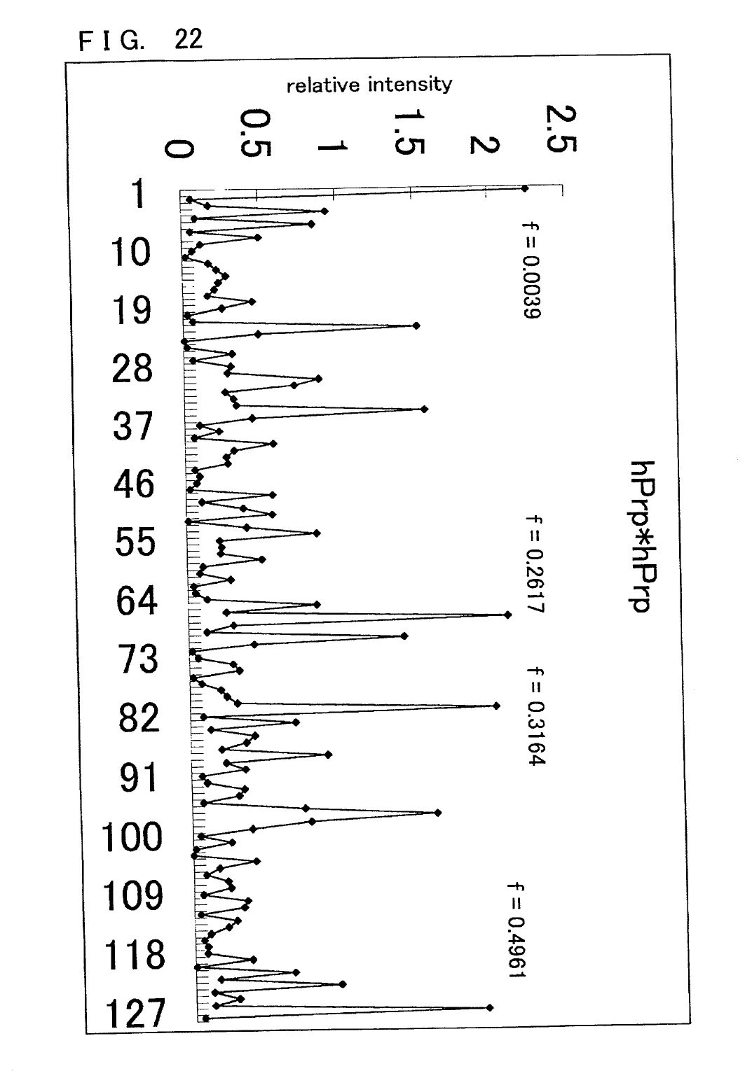

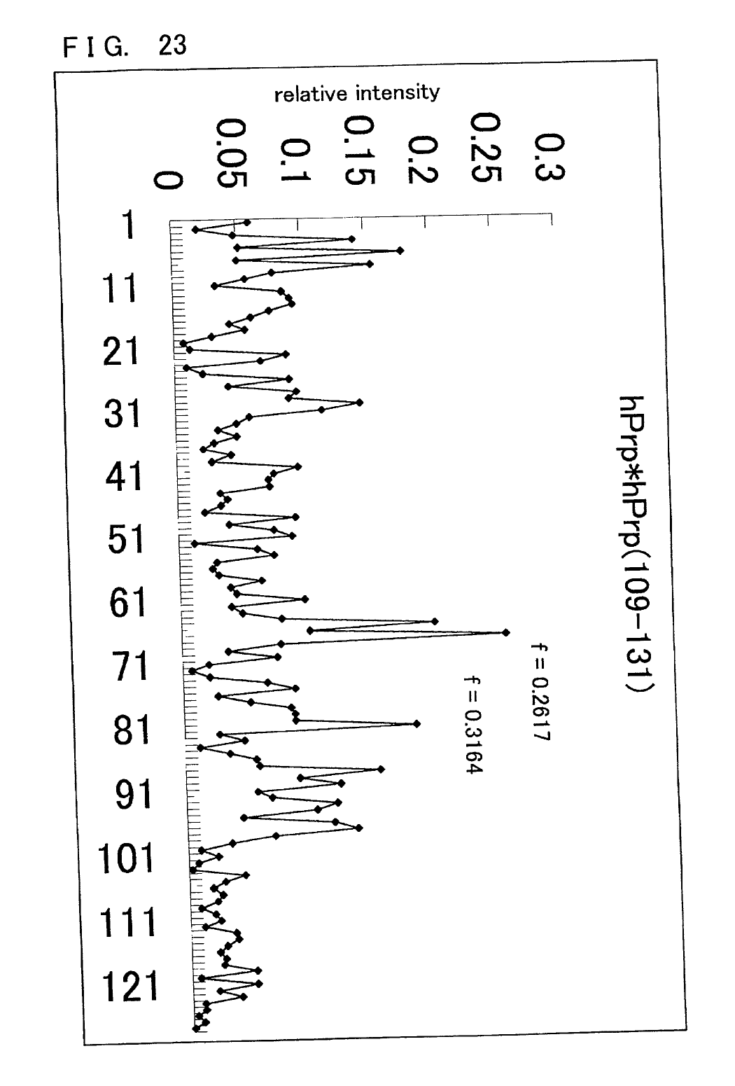

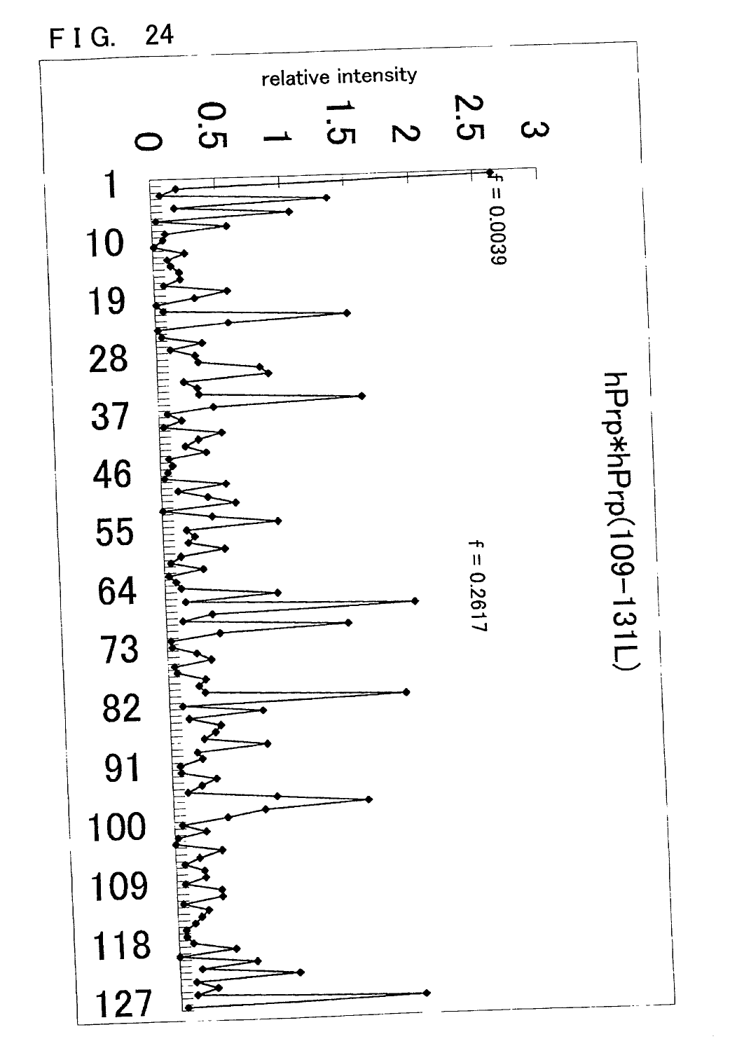

Biological activity of normal prion protein has hitherto not been reported (S. B. Prusiner, Proc. Natl. Acad. Sci., U.S.A., 95, 13363-13383 (1998); D. Westway et al., Proc. Natl. Acad. Sci., U.S.A., 95, 11030-11031 (1998)). The amino acid sequence has already been registered in SWISS-PROT. According to a known literature (G. Forloni et al., Nature, 362, 543-546 (1993)), the neurotoxic activity is known to exist at around amino acid numbers of 106 to 126 of the prion protein. However, there is a counterevidence that the peptide does not exhibit neurotoxic activity when the peptide of the sequence is treated at 37° C. for 30 days in a buffer solution (pH 7.4) (B. Kunz et al., FEBS Lett., 458, 65-68 (1999)). The present inventor has first determined a self-cross-spectrum of the total amino acid sequence frequency spectrum of the prion and a cross-spectrum of the total amino acid sequence frequency spectrum of the prion and a frequency spectrum of the active site (amino acid numbers of 109 to 131). Then, the total amino acid sequence frequency spectrum of the prion and a replaced total amino acid sequence frequency spectrum of the prion wherein the amino acid residues in the active site region was replaced by leucine were crossed (FIGS. 22, 23, and 24). The results are shown in Table 7.

TABLE 7
Prion
Frequency values derived from total sequence (256)
0.0039 0.2617 0.3164 0.4961, 0.3789
Frequency values derived from active site (256)
0.2617 0.2539 0.3164 0.0234 0.0313
Frequency values I of replaced total sequence (256)
0.0039 0.2617 0.4961 0.3614 0.1367

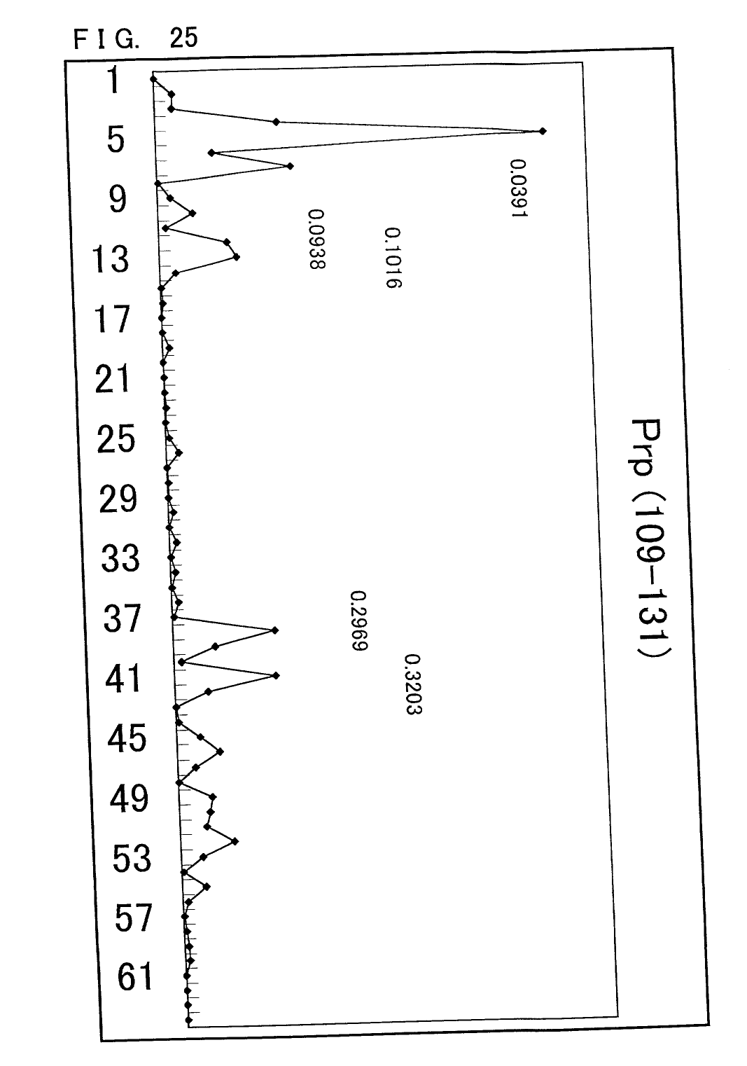

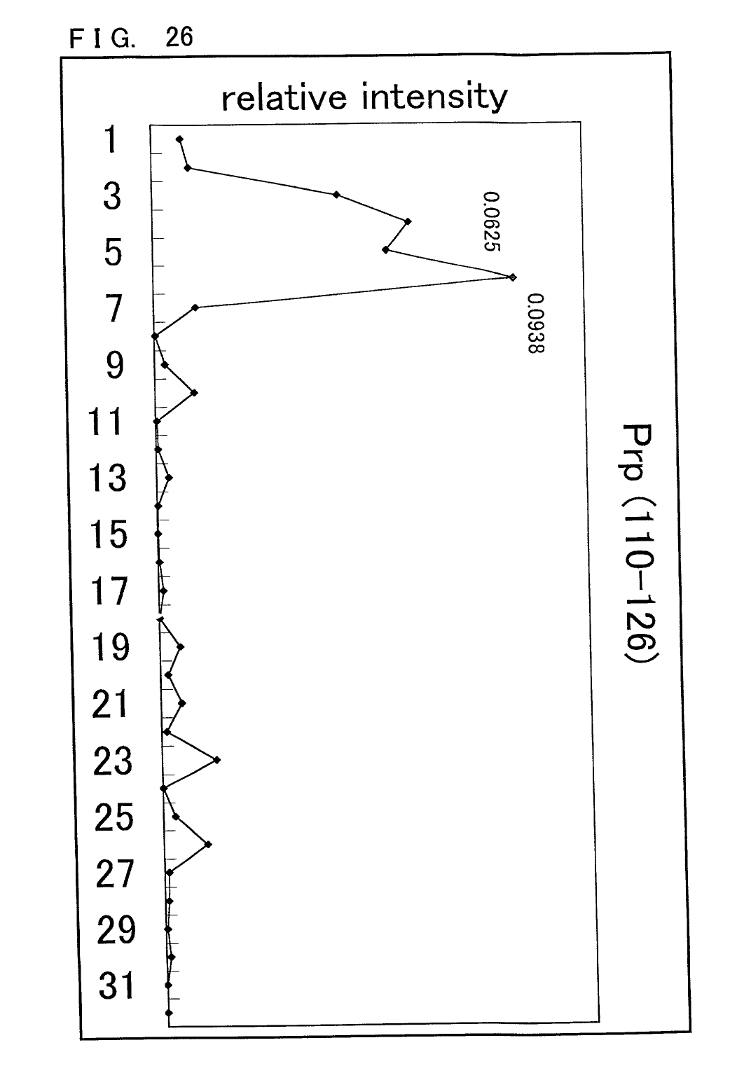

From Table 7, the characteristic frequency values of 0.2617, 0.2539, 0.0234, 0.0313, and so forth are peaks derived from the region of 109 to 131. These values are reserved also in a cross-spectrum of the total amino acid sequence frequency spectrum of the prion and the total amino acid sequence frequency spectrum of a magainin 2 derivative (MSI-78A). Accordingly, biological•functional activity similar to MSI-78A can be expected at the region of 109 to 131. Furthermore, in order to examine the prediction, a mixed spectrum (FIG. 25) of the region of 109 to 131 of the prion protein was compared with the mixed spectrum (FIG. 7) of MSI-78A. From FIG. 25, among the values of 0.0391, 0.0547, 0.0313, 0.3203, 0.2969, 0.1016, 0.0938, etc. selected as prominent frequency values, 0.1016 and 0.0938 were found to be coincident with or close to 0.0938 of MSI-78A. Among the two frequency values, 0.0938 was suggested to be the most prominent value in the mixed spectrum (FIG. 26) of the region of 106 to 131 of the prion protein. From these results, decarboxylation activity may be expected not only in the region of 106 to 126 present in the amino acid sequence of the region of 109 to 131 but also as one biological activity of the whole normal prion protein molecule. Furthermore, based on a known literature (I. Cosic, IEEE, 41, 1101-1114 (1994)), the frequency values of 0.0625 and 0.0781 are close to the frequency values of myoglobin, cytochrome, or the like. In fact, it has been reported that the prion protein is a copper-binding protein and takes part in biological reaction as an antioxidant (D. R. Brown et al., J. Neurochem., 76, 69-76 (2001)).

### EXAMPLE 2

Prediction of Function of an Amyloid Protein precursor (APP)

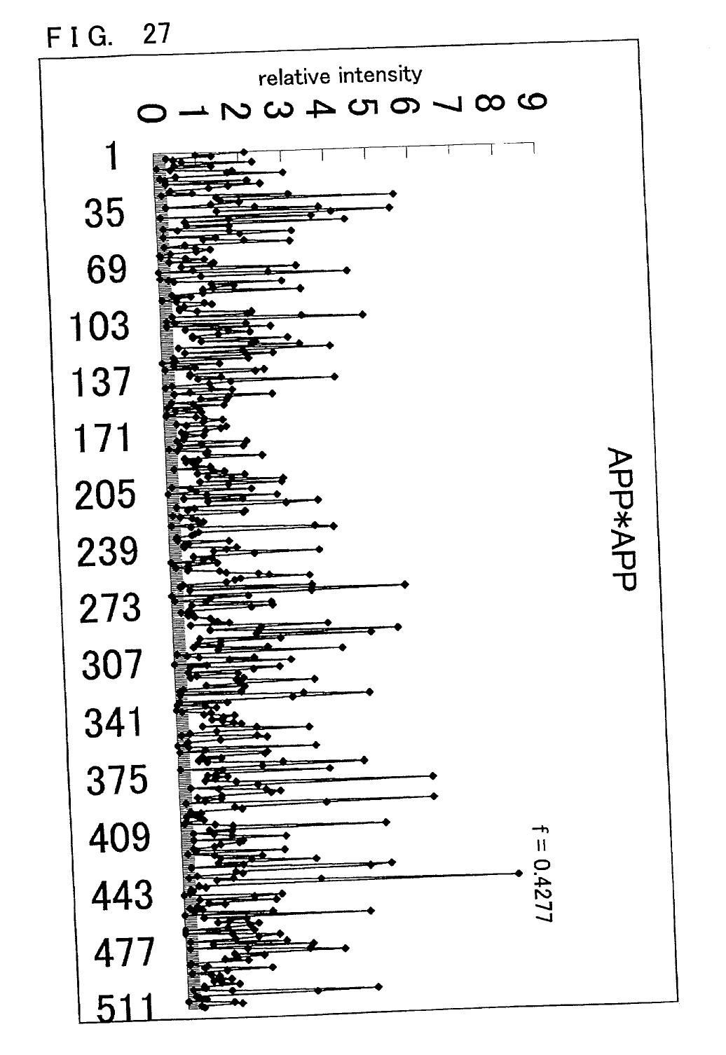

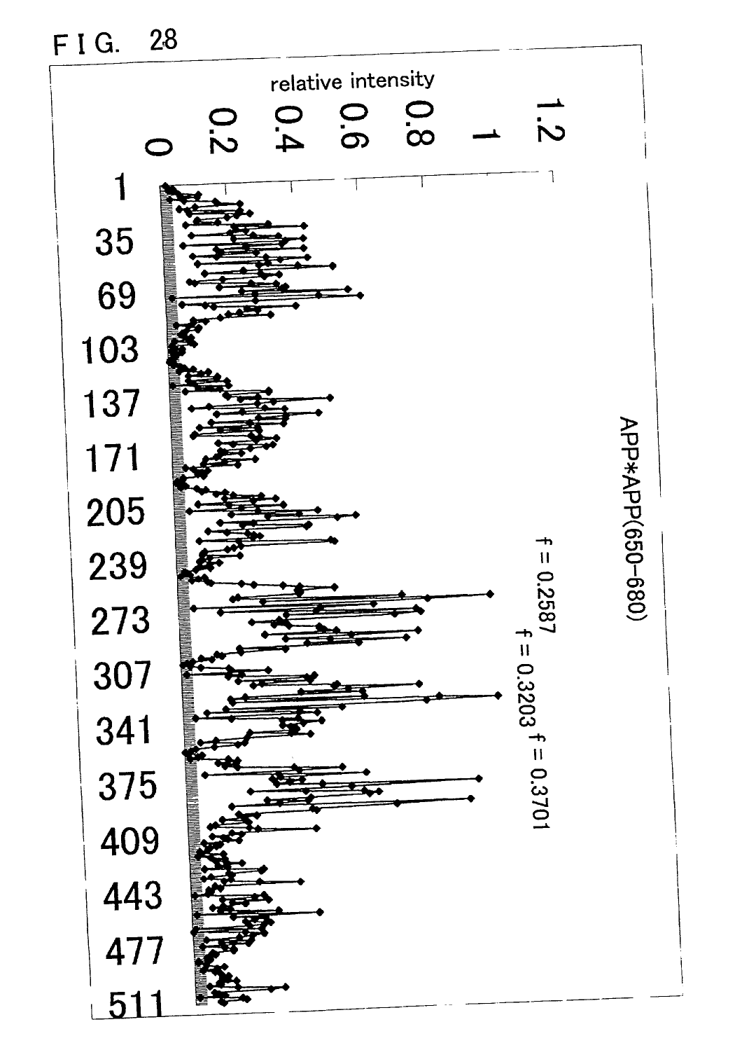

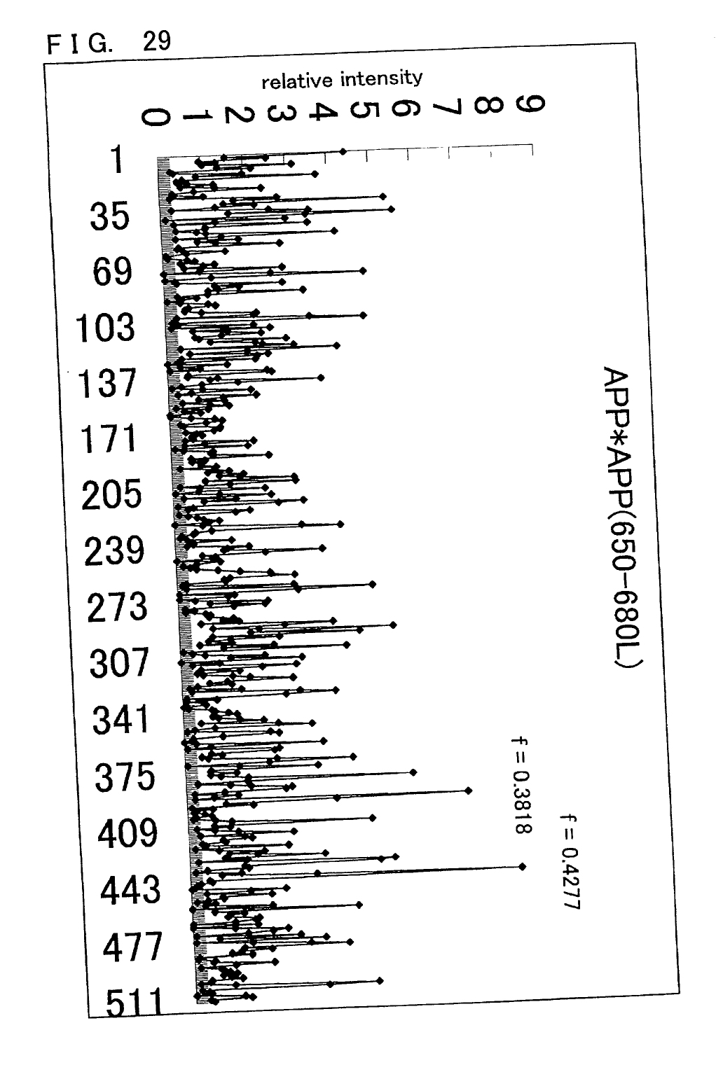

Among three types of APP, one is a protein comprising 751 amino acids (P. Ponte et al., Nature, 331, 525-527 (1988)). The amino acid sequence has already been registered in SWISS-PROT. It has already been predicted that functionally active sites of APP exist at the periphery of amino acid numbers of 142, 340, 513, and 655 in the method for predicting a functional site of a protein (N. Numao et al., Biol. Pharm. Bull., 16, 1160-1163 (1993)). Among them, the biological activity of the region (amino acid numbers of 650 to 680) containing motifs (VXH, KL, and GA) has been reported to be particularly pertinent to senile dementia. The present inventor examined the present method for searching for a novel biological•functional activity of the region. That is, assuming the region as an active site, the examination was conducted according to the above method (FIGS. 27, 28, and 29). The results are shown in Table 8.

TABLE 8
APP
Frequency values derived from total sequence (1024)
0.4277 0.3818 0.3701 0.0283 0.3610
Frequency values derived from active site (1024)
0.3203 0.2588 0.3701 0.3818 0.3193
Frequency values I of replaced total sequence (1024)
0.4277 0.3818 0.0361 0.3701 0.0283

From Table 8, the frequency values derived from the active site of APP was suggested to be from 0.3193 to 0.3203 and 0.2588. The values of 0.3193 to 0.3203 are close to glucagon (0.3203±0.034) and lysozyme (0.32811±0.004).

### EXAMPLE 3

Prediction of Activity of the Active Region of Inhibiting a Protease in APP

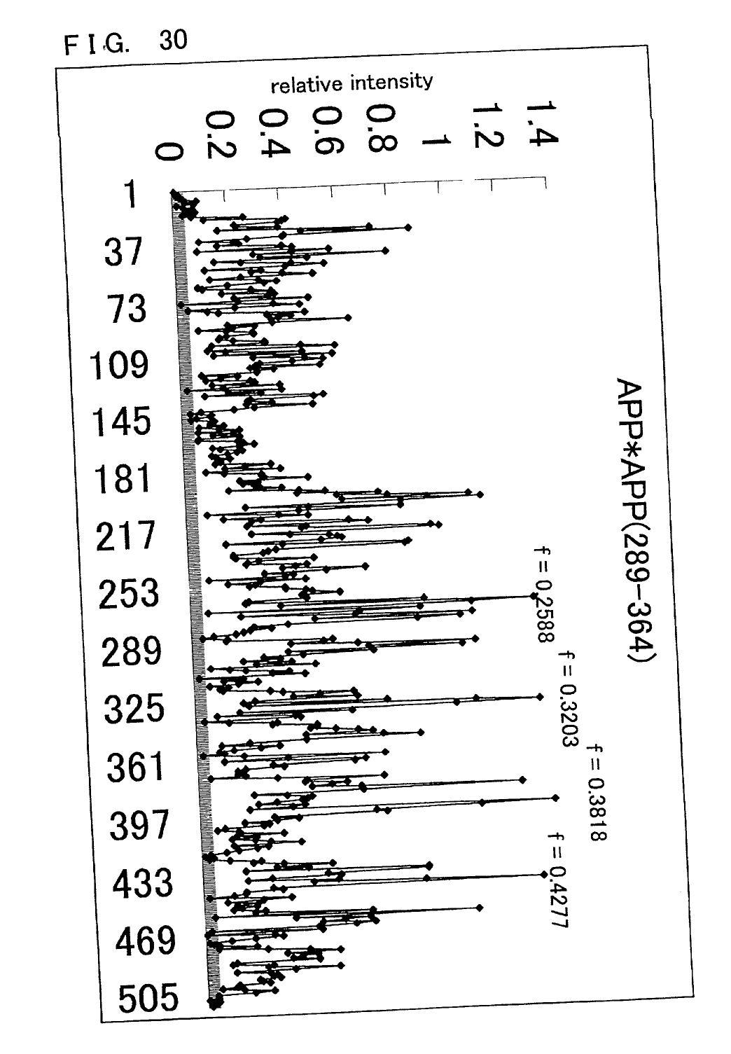

It is already known that the amino acid sequence in the periphery of 291 to 341 is highly homologous to serine protease inhibitor (P. Ponte et al., Nature, 331, 525-527 (1988)). In fact, the inhibitory activity of this region has already been reported (N. Kitaguchi et al., Nature, 331, 530-532 (1988)), but the inhibitory activity is not high. With reference to the experimental results, the present method was applied using the region of amino acid numbers of 289 to 364 as an active site region and operation was attempted (FIG. 30). The results are shown in Table 9.

TABLE 9
Prediction of activity in the region of 289 to 364 in APP
Frequency values derived from total sequence (1024)
0.4277 0.3818 0.3701 0.0283 0.3610
Frequency values derived from active site (1024)
0.3818 0.3203 0.2587 0.4277 0.3701

According to a known literature (I. Cosic, IEEE, 41, 1101-1114 (1994)), the frequency value of the protease inhibitor is 0.3555+0.008, so that the above values of top 2 are not coincident. Incidentally, when operation on the kunitz protease inhibitors described in a known literature (P. Ponte et al., Nature, 331, 525-527 (1988)), characteristic frequency value thereof was 0.3281. Among the above 5 values, 0.3203 is the most closest value. Therefore, the inhibitory activity may be expected as a novel biological activity of the region of 650 to 680 in APP of Example 2.

### EXAMPLE 4

Prediction of Novel Biological Activity of Human Growth Hormone (hGH)

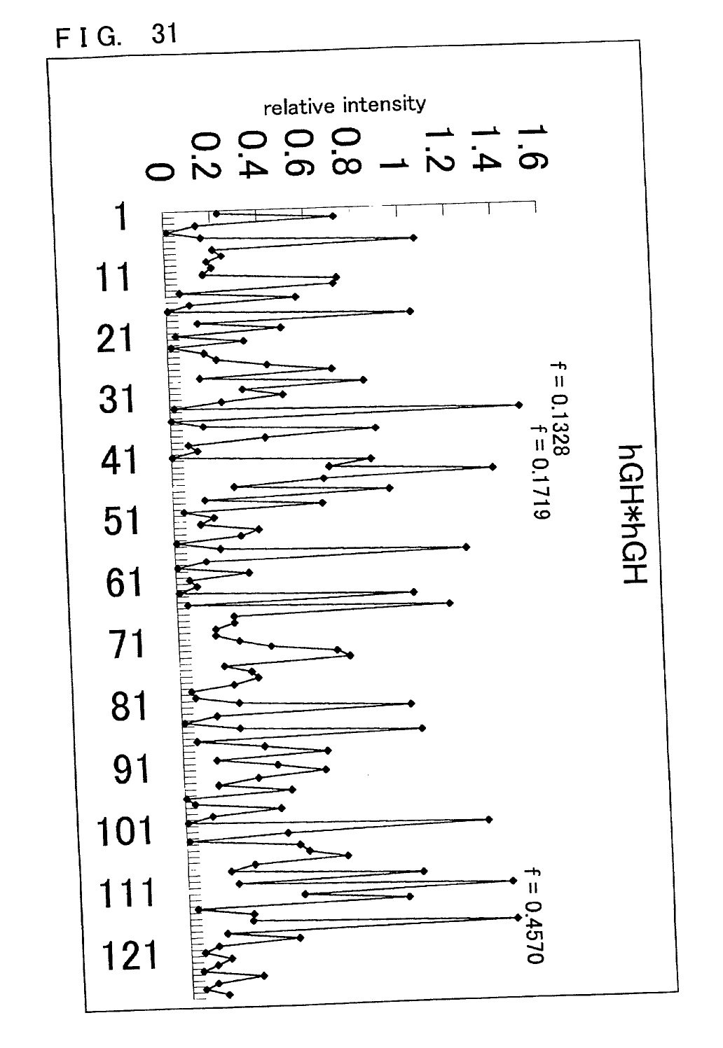

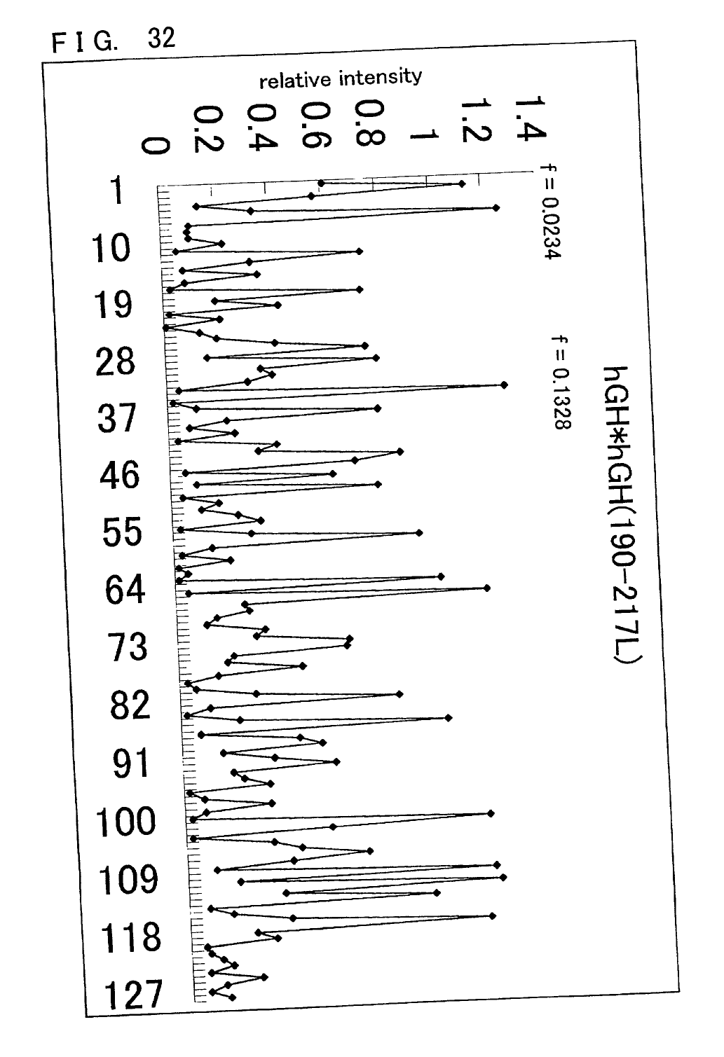

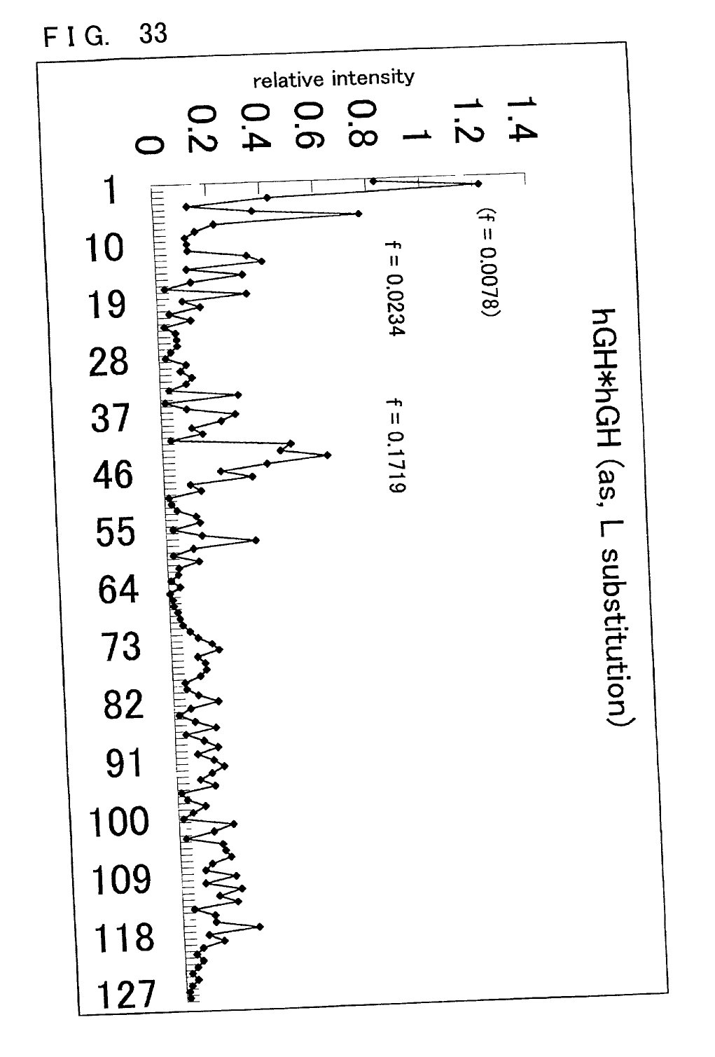

On human growth hormone (hGH) comprising 217 amino acids and having protein synthetic, cartilage growth promoting and lipocatabolic actions, it was examined according to the present method whether a novel biological•functional activity derived from an active site was expectable or not. The amino acid sequence has already been registered in SWISS-PROT. Since the active site was predicted to exist in the periphery of amino acid number of 205 (N. Numao et al., Biol. Pharm. Bull., 16, 1160-1163 (1993)), the present method was applied in a similar manner to Example 2 with reference to the above prediction. That is, the examination was conducted using the region of amino acid number of 197 to 217 as an active site (FIGS. 31, 32, and 33). The results are shown in Table 10.

TABLE 10
Human growth hormone
Frequency values derived from total sequence (256)
0.1328 0.4570 0.4336 0.1719 0.3945
Frequency values I of replaced total sequence (256)
0.1328 0.0234 0.2578 0.4336 0.4258
Frequency values II of replaced total sequence (256)
0.0234 0.1719 0.1641 0.1680 0.0508

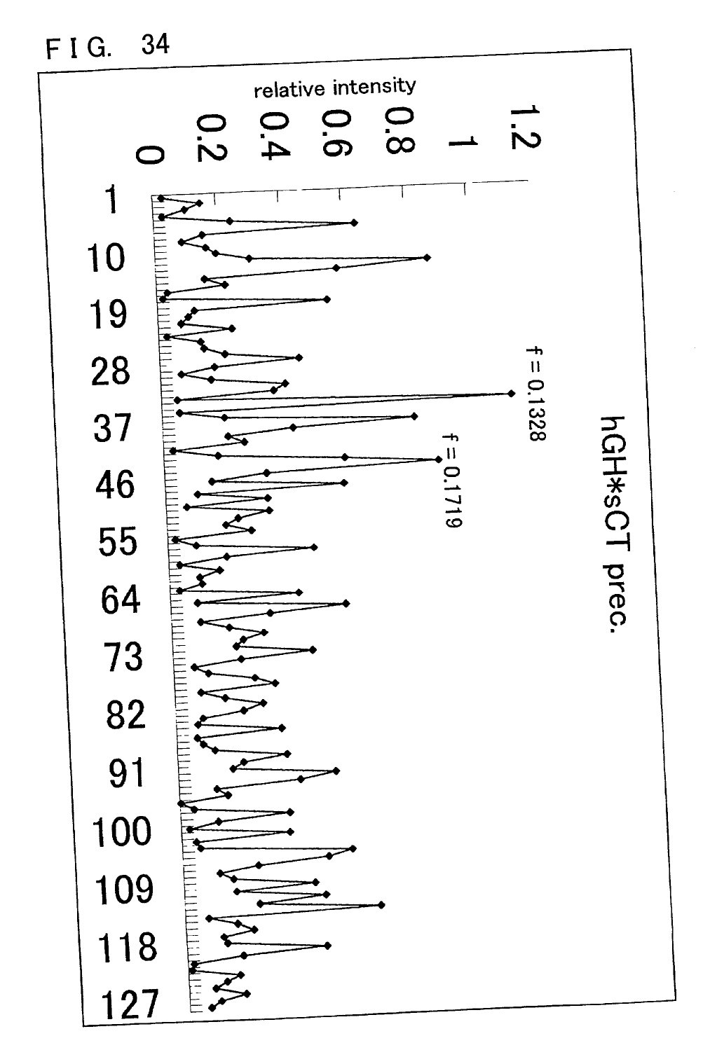

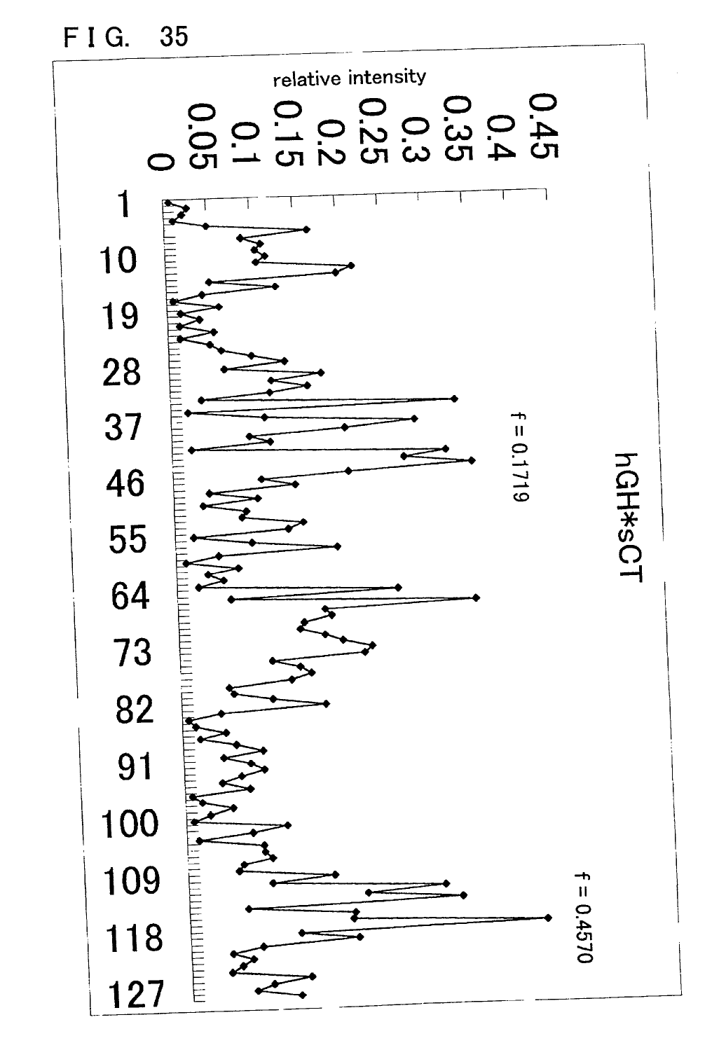

From Table 10, the biological activity dependent on the active site of hGH relates to the frequency values of 0.0234 and 0.1641 to 0.1719, but the former overlaps with the frequency value other than the active site region. Therefore, the activity is derived from the whole molecule. In fact, it is already known that three active sites are present in hGH (B. C. Cunningham et al, Science, 244, 1081-1085 (1989)). However, the frequency values of 0.1641 to 0.1719 are near to the frequency values derived from the active site of salmon calcitonin (0.1445 to 0.1563). Thus, the total amino acid sequence frequency spectrum of hGH and the frequency spectrum of salmon calcitonin precursor or the frequency spectrum of salmon calcitonin comprising 32 amino acids were crossed (FIGS. 34 and 35). As a result, the prominence of the peaks at 0.1328 to 0.1719 was observed. Accordingly, a similar biological activity between hGH and salmon calcitonin is expectable.

### EXAMPLE 5

Construction of Database

There may be various methods for constructing a database with regard to biological functions derived from active sites of proteins. For instance, the method comprises summarizing the results (Tables 1 to 10) obtained in the present invention for the purpose of easy searching by means of a computer. Table 11 shows one example.

TABLE 11

| | | | | | |
|---|---|---|---|---|---|
| Magainin Activity | 0.4355 | 0.4336 | 0.0645 | 0.0664 | 0.2598 |
| Salmon calcitonin | 0.1563 | 0.2734 | 0.1445 | 0.0469 | 0.1523 |
| Activity | | | | | |
| Gamma interferon | 0.0234 | 0.3594 | 0.3633 | 0.0273 | 0.4023 |
| Activity | | | | | |
| APP Activity | 0.3203. | 0.2588 | 0.3701 | 0.3818 | 0.3193 |
| (amyloid region) | | | | | |
| Activity | 0.3818 | 0.3203 | 0.2587 | 0.4277 | 0.3701 |
| (inhibitory region) | | | | | |
| Human growth | 0.0234 | 0.1718 | 0.1641 | 0.1680 | 0.0508 |
| hormone Activity | | | | | |

Thus, it is easily conceived that a useful database extremely superior to the conventional prediction of functions of proteins can be constructed by operating on various proteins according to the present method and adding separately the results of activity evaluation. Accordingly, as far as the fundamental concept of the present invention is utilized, the construction of a database based on the results even for proteins other than those described in the present specification falls within the range of the present claims.

### EXAMPLE 6

Utilization of Database

From the database of Table 11, decarboxylation activity may be also expected in APP. The region of 650 to 680 of APP and the prion protein are expectable to have a protease inhibitory activity. The calcitonin and hGH are expected to have a similar activity. Also, hGH and gamma interferon are expected to have a similar activity.

### EXAMPLE 7

Prediction of Ebola Virus Binding Protein

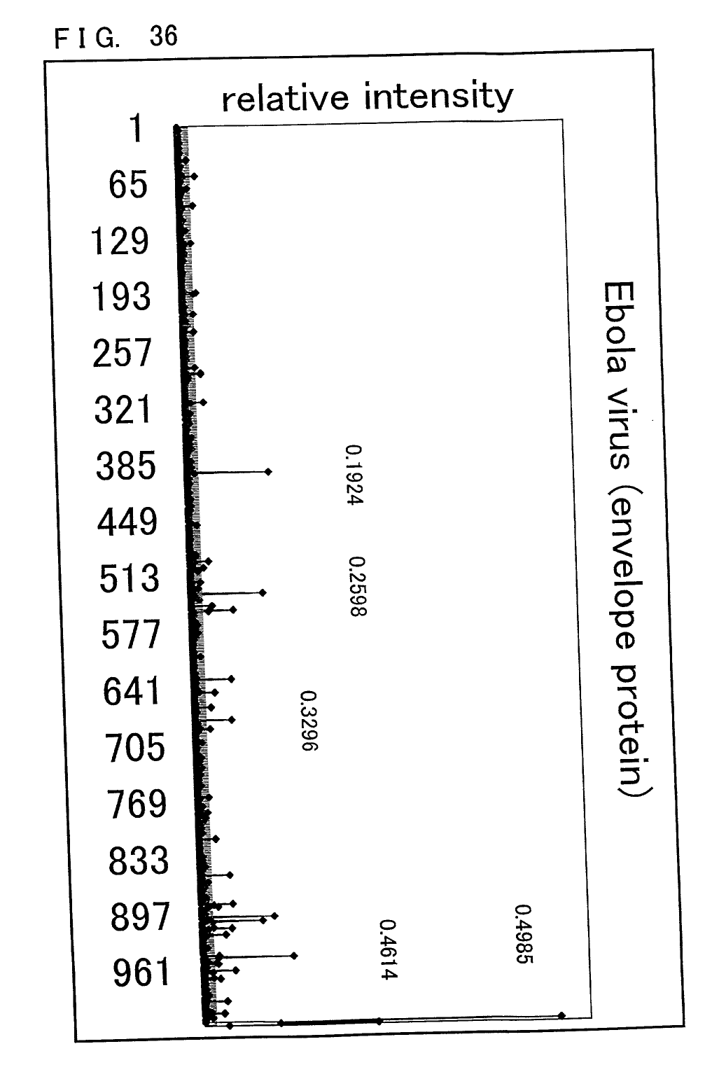

Ebola virus is known to be one of international infectious diseases, which causes viral hemorrhagic fever. However, the receptor protein has still not been reported. When prediction of binding activity between several receptor proteins employed in the present invention and the envelope protein of Ebola virus (FIG. 36) (V. E. Volchkov et al., Virology 214, 421-430 (1995)) was conducted, among CD4 receptor, poliovirus receptor, IL−2 receptor, 55kd TNF receptor, and insulin receptor, the interaction with the extracellular region of 55kd TNF receptor was more highly predicted than the cases of other receptors.

In contrast to the conventional activity evaluating methods which are near to a random process, the present method is extremely useful since it can predict a novel functional activity or binding partner of an arbitrary protein or nucleotide sequence based on databases of proteins and nucleotide sequences known beforehand. (Example 8) Construction and Application of Database Based on the method of this invention, the following database (Table 12) of proteins involved in phosphates system in Saccharomyces cerrevisiae is conveniently constructed in order to predict the specific protein-protein interaction.

TABLE 12

| | pho2 | pho4 | pho5 | pho8 | pho80 | pho81 | pho86 | pho87 | pho88 | pho89 |
|---|---|---|---|---|---|---|---|---|---|---|
| pho 2 | 2 | 3 | 0 | 1 | 0 | 0 | 1 | 1 | 3 | 1 |
| pho 4 | 3 | 9 | 1 | 1 | 0 | 1 | 2 | 1 | 1 | 1 |
| pho 5 | 0 | 1 | 3 | 1 | 0 | 2 | 2 | 4 | 1 | 1 |
| pho 8 | 1 | 0 | 1 | 0 | 1 | 1 | 1 | 1 | 1 | 0 |
| pho80 | 0 | 0 | 0 | 1 | 2 | 0 | 0 | 0 | 1 | 1 |
| pho81 | 2 | 1 | 2 | 2 | 0 | 1 | 3 | 1 | 2 | 2 |
| pho84 | 1 | 2 | 2 | 2 | 0 | 2 | 2 | 3 | 4 | 2 |
| pho85 | 0 | 1 | 2 | 1 | 0 | 2 | 1 | 3 | 2 | 1 |
| pho86 | 1 | 2 | 2 | 1 | 0 | 1 | 0 | 4 | 3 | 1 |
| pho87 | 1 | 1 | 3 | 1 | 0 | 3 | 4 | 2 | 2 | 3 |
| pho88 | 3 | 1 | 1 | 1 | 1 | 2 | 3 | 2 | 2 | 4 |
| pho89 | 1 | 1 | 1 | 0 | 1 | 1 | 1 | 2 | 4 | 3 |

The number is shown of resonant frequencies between two proteins in the matrix.

As shown in the previous reports (Y. Ohshima Genes, Gnet. Syst. 72, 323-334 (1997); N. Ogawa, J. DeRisi, P.O. Brown, Molecular Biology of the Cell 11, 4390-4321 (2000)), pho4 is a positive regurator, and necessary for the transcription activation in the phosphates system. Based on Table 12, the pho81, pho85, pho87, pho88 or pho89 can be selected under the definite conditions of minimum number (resonant frequency =1) and the local existence of proteins in the cell. According to the previous study, pho4 has been reported to interact with pho85 and pho81. The other interaction remains to open. Therefore, this inventive method will be clearly useful to predict and find a novel intermolecular interaction in various proteins without using 3D structure.

TABLE 13
The prediction of interaction between various proteins
involved in apoptosis

| | TNF-α | TNFR out | TNFR in | FASL | FAS out | FAS in | TRADD | FADD |
|---|---|---|---|---|---|---|---|---|
| tnf-α | 0 | 1 | 0 | 0 | 0 | 0 | 0 | 1 |
| tnfr out | 1 | 2 | 0 | 0 | 2 | 2 | 1 | 2 |
| tnfr in | 0 | 0 | 0 | 1 | 1 | 0 | 1 | 0 |
| fasL | 0 | 0 | 1 | 0 | 1 | 3 | 0 | 0 |
| fas out | 0 | 2 | 1 | 1 | 2 | 1 | 3 | 2 |
| fas in | 0 | 2 | 0 | 2 | 1 | 5 | 4 | 2 |
| tradd | 0 | 1 | 1 | 0 | 3 | 4 | 0 | 1 |
| fadd | 1 | 3 | 0 | 0 | 2 | 2 | 1 | 2 |
| casp 8 | 0 | 1 | 3 | 2 | 1 | 5 | 3 | 1 |
| rip | 0 | 0 | 1 | 1 | 1 | 3 | 2 | 1 |
| raidd | 0 | 1 | 2 | 2 | 0 | 1 | 2 | 2 |
| casp 2 | 0 | 0 | 1 | 1 | 0 | 1 | 1 | 0 |
| casp 3 | 0 | 0 | 2 | 1 | 1 | 3 | 1 | 0 |
| sodd | 0 | 0 | 0 | 1 | 1 | 1 | 1 | 0 |
| flip long | 1 | 0 | 0 | 1 | 1 | 2 | 1 | 0 |
| flip short | 2 | 0 | 0 | 1 | 0 | 2 | 0 | 1 |

| | CASP 8 | RIP | RAIDD | CASP 2 | CASP 3 | SODD | FLIPlong | FLIPshort |
|---|---|---|---|---|---|---|---|---|
| tnf-α | 0 | 0 | 0 | 0 | 0 | 0 | 1 | 2 |
| tnfr extra | 2 | 0 | 1 | 0 | 0 | 0 | 0 | 0 |
| tnfr intra | 3 | 1 | 2 | 1 | 2 | 0 | 0 | 0 |
| fasL | 2 | 1 | 2 | 1 | 1 | 1 | 1 | 1 |
| fas extra. | 1 | 1 | 0 | 0 | 1 | 1 | 1 | 0 |
| fas intra. | 4 | 3 | 1 | 1 | 3 | 1 | 2 | 2 |
| tradd | 3 | 2 | 2 | 1 | 1 | 1 | 1 | 0 |
| fadd | 1 | 1 | 2 | 0 | 0 | 0 | 0 | 1 |
| casp 8 | 0 | 1 | 0 | 1 | 1 | 2 | 3 | 3 |
| rip | 1 | 1 | 1 | 1 | 2 | 2 | 1 | 0 |
| raidd | 0 | 1 | 0 | 0 | 1 | 2 | 0 | 1 |
| casp 2 | 1 | 1 | 0 | 0 | 1 | 1 | 0 | 0 |
| casp 3 | 1 | 2 | 1 | 1 | 1 | 1 | 1 | 1 |
| sodd | 3 | 2 | 2 | 1 | 1 | 2 | 2 | 2 |
| flip long | 3 | 1 | 0 | 0 | 1 | 2 | 2 | 1 |
| flip short | 3 | 0 | 1 | 0 | 1 | 2 | 1 | 0 |

Table 13 shows the number of characteristic frequencies of the interaction between typical proteins involved in apoptosis ( Nagata S., Cell 88, 355-365 (1997)) by this innovation method. The number means the resonant number of between two proteins. Of various numbers, number 1 predict higher probability of protein-protein interaction than other interaction in each column. For example, TNF-α likes to interact with TNFR out (TNFR extracellular region) more than the TNFR in (TNFR intracellular region). In this, TNF-α cannot interact with FADD or FLIP short, because the latters are cytoplasmic proteins. Also, Fas ligand (FASL) can interact with FAS out (FAS extracellular region), but not with TNFR out (TNFR extracellular region).

What is claimed is:

1. A method for predicting biological•functional activity and/or binding activity of an arbitrary protein, which comprises:
determining a total amino acid sequence frequency spectrum obtained by giving EIIP (Electron-ion interaction potential) index values to the amino acid residues of an arbitrary amino acid sequence originated in natural-type or non-natural-type and subjecting the resulting numerical value sequence (EIIP sequence) to discrete Fourier transformation (DFT), and
an active site frequency spectrum obtained by giving EIIP index values to the amino acids of an amino acid sequence region, which is composed of 2 to 64 amino acid residues present in the above arbitrary amino acid sequence and contains at least one known motif pertinent to an active site and subjecting the resulting EIIP sequence to DFT; and
selecting one or more characteristic frequency values derived from an active site of the protein from the cross-spectrum of the above total amino acid sequence frequency spectrum and the above active site frequency spectrum, and searching for one or more approximate frequency values of well-known characterized proteins similar to the characteristic frequency values described above.

2. The method for predicting biological•functional activity and/or binding activity of an arbitrary protein according to claim 1, wherein as the known motif as a signal of the active site, any one or more of GT, AS, GA, ID, TR, SR, LK, TXW, VXH, MXH, WXP, AXC, GXS (wherein G, T, A, S, I, D, R, L, K, W, V, H, M, P, C, and X mean glycine, threonine, alanine, serine, isoleucine, aspartic acid, arginine, leucine, lysine, tryptophan, valine, histidine, methionine, proline, cysteine, and any of 20 kinds of amino acids, respectively) and/or the reversed sequences thereof are employed.

3. A method for predicting biological•functional activity and/or binding activity of an arbitrary protein, which comprises:
determining a total amino acid sequence frequency spectrum obtained by giving EIIP (Electron-ion interaction potential) index values to the amino acid residues of an arbitrary amino acid sequence originated in natural-type or non-natural-type and subjecting the resulting numerical value sequence (EIIP sequence) to discrete Fourier transformation (DFT), and
a total nucleotide sequence frequency spectrum obtained by giving EIIP index values to the nucleotide residues of a nucleotide sequence region academically corresponding to the above amino acid sequence and subjecting the resulting EIIP sequence to DFT; and
selecting one or more characteristic frequency values derived from the protein from the cross-spectrum of the above total amino acid sequence frequency spectrum and the above active site frequency spectrum, and searching for one or more approximate frequency values of well-known characterized proteins similar to the characteristic frequency values described above.

4. A method for predicting biological•functional activity and/or binding activity of an arbitrary nucleotide sequence, which comprises:
determining first total nucleotide sequence frequency spectrum obtained by giving EIIP (Electron-ion interaction potential) index values to the nucleotide residues of an arbitrary single-strand nucleotide sequence originated in natural-type or non-natural-type and subjecting the resulting numerical value sequence (EIIP sequence) to discrete Fourier transformation (DFT), and
second total nucleotide sequence frequency spectrum obtained by giving EIIP index values to the nucleotide residues of a nucleotide sequence which binds to the above nucleotide sequence through hydrogen bonding, and subjecting the resulting EIIP sequence to DFT; and
selecting one or more characteristic frequency values derived from the nucleotide sequence from the cross-spectrum of the above first total nucleotide sequence frequency spectrum and the above second nucleotide sequence frequency spectrum, and searching for one or more approximate frequency values of well-known characterized proteins similar to the characteristic frequency values described above.

5. A method for predicting biological•functional activity and/or binding activity of an arbitrary amino acid sequence originated in natural-type or non-natural-type and other nucleotide sequence, which comprises:
determining at least two spectra of the following five spectra:
first spectrum of a total amino acid sequence frequency spectrum obtained by giving EIIP (Electron-ion interaction potential) index values to the amino acid residues of an arbitrary amino acid sequence originated in natural-type or non-natural-type and subjecting the resulting numerical value sequence (EIIP sequence) to discrete Fourier transformation (DFT),
second spectrum of an active site frequency spectrum obtained by giving EIIP index values to the amino acids of an amino acid sequence region, which is composed of 2 to 64 amino acid residues present in an arbitrary amino acid sequence and contains at least one known motif pertinent to an active site and subjecting the resulting EIIP sequence to DFT,
third spectrum of a total nucleotide sequence frequency spectrum obtained by giving EIIP index values to the nucleotide residues of a nucleotide sequence region academically corresponding to the amino acid sequence and subjecting the resulting EIIP sequence to DFT,
fourth spectrum of a total nucleotide sequence frequency spectrum obtained by giving EIIP index values to the nucleotide residues of an arbitrary single-strand nucleotide sequence originated in natural-type or non-natural-type and subjecting the resulting EIIP sequence to DFT, and
fifth spectrum of a total nucleotide sequence frequency spectrum obtained by giving EIIP index values to the nucleotide residues of a complementary nucleotide sequence which binds to a nucleotide sequence through hydrogen bonding, and subjecting the resulting EIIP sequence to DFT; and
comparing with each spectrum.

6. The method for predicting biological•functional activity and/or binding activity of an arbitrary amino acid sequence and other nucleotide sequence according to claim 5, wherein as the known motif as a signal of the active site, any one or more of GT, AS, GA, ID, TR, SR, LK, TXW, VXH, MXH, WXP, AXC, GXS (wherein G, T, A, S, I, D, R, L, K, W, V, H, M, P, C, and X mean glycine, threonine, alanine, serine, isoleucine, aspartic acid, arginine, leucine, lysine, tryptophan, valine, histidine, methionine, proline, cysteine, and any of 20 kinds of amino acids, respectively) and/or reverse sequences thereof are employed.

7. A method for predicting an active site of an arbitrary amino acid sequence or nucleotide sequence originated in natural-type or non-natural-type, which comprises:
determining at least two spectra of the following five spectra:
first spectrum of a total amino acid sequence frequency spectrum obtained by giving EIIP (Electron-ion interaction potential) index values to the amino acid residues of an arbitrary amino acid sequence originated in natural-type or non-natural-type and subjecting the resulting numerical value sequence (EIIP sequence) to discrete Fourier transformation (DFT),
second spectrum of an active site frequency spectrum obtained by giving EIIP index values to the amino acids of an amino acid sequence region, which is composed of 2 to 64 amino acid residues present in an arbitrary amino acid sequence and contains at least one known motif pertinent to an active site and subjecting the resulting EIIP sequence to DFT,
third spectrum of a total nucleotide sequence frequency spectrum obtained by giving EIIP index values to the nucleotide residues of a nucleotide sequence region academically corresponding to the amino acid sequence and subjecting the resulting EIIP sequence to DFT,
fourth spectrum of a total nucleotide sequence frequency spectrum obtained by giving EIIP index values to the nucleotide residues of an arbitrary single-strand nucleotide sequence originated in natural-type or non-natural-type and subjecting the resulting EIIP sequence to DFT, and
fifth spectrum of a total nucleotide sequence frequency spectrum obtained by giving EIIP index values to the nucleotide residues of a complementary nucleotide sequence which binds to a nucleotide sequence through hydrogen bonding, and subjecting the resulting EIIP sequence to DFT; and
comparing with each spectrum.

8. The method for predicting an active site of an arbitrary amino acid sequence or nucleotide sequence according to claim 7, wherein as the known motif as a signal of the active site, any one or more of GT, AS, GA, ID, TR SR, LK, TXW, VXH, MXH, WXP, AXC, GXS (wherein G, T, A, S, I, D, R, L, K, W, V, H, M, P, C, and X mean glycine, threonine, alanine, serine, isoleucine, aspartic acid, arginine, leucine, lysine, tryptophan, valine, histidine, methionine, proline, cysteine, and any of 20 kinds of amino acids, respectively) and/or reverse sequences thereof are employed.

9. A method for predicting biological•functional activity and/or binding activity of an arbitrary amino acid sequence and/or an arbitrary nucleotide sequence, which comprises:
determining at least two spectra of the following five spectra:
first spectrum of a total amino acid sequence frequency spectrum obtained by giving EIIP (Electron-ion interaction potential) index values to the amino acid residues of an arbitrary amino acid sequence originated in natural-type or non-natural-type and subjecting the resulting numerical value sequence (EIIP sequence) to discrete Fourier transformation (DFT),
second spectrum of an active site frequency spectrum obtained by giving EIIP index values to the amino acids of an amino acid sequence region, which is composed of 2 to 64 amino acid residues present in an arbitrary amino acid sequence and contains at least one known motif pertinent to an active site and subjecting the resulting EIIP sequence to DFT,
third spectrum of a total nucleotide sequence frequency spectrum obtained by giving EIIP index values to the nucleotide residues of a nucleotide sequence region academically corresponding to the amino acid sequence and subjecting the resulting EIIP sequence to DFT,
fourth spectrum of a total nucleotide sequence frequency spectrum obtained by giving EIIP index values to the nucleotide residues of an arbitrary single-strand nucleotide sequence originated in natural-type or non-natural-type and subjecting the resulting EIIP sequence to DFT, and
fifth spectrum of a total nucleotide sequence frequency spectrum obtained by giving EIIP index values to the nucleotide residues of a nucleotide sequence which binds to the nucleotide sequence through hydrogen bonding, and subjecting the resulting EIIP sequence to DFT; and
comparing with each spectrum.

10. The method for predicting biological•functional activity and/or binding activity of an arbitrary amino acid sequence and/or an arbitrary nucleotide sequence according to claim 9, wherein as the known motif as a signal of the active site, any one or more of GT, AS, GA, ID, TR SR, LK, TXW, VXH, MXH, WXP, AXC, GXS (wherein G, T, A, S, I, D, R, L, K, W, V, H, M, P, C, and X mean glycine, threonine, alanine, serine, isoleucine, aspartic acid, arginine, leucine, lysine, tryptophan, valine, histidine, methionine, proline, cysteine, and any of 20 kinds of amino acids, respectively) and/or reverse sequences thereof are employed.

11. A program wherein the method according to any one of claims 1 to 10 is constructed using a mathematical means, which realizes, on a computer, a function capable of predicting novel biological•functional and/or binding activity of desired protein, amino acid sequence, or nucleotide sequence.

12. The program according to claim 11, wherein the above mathematical means is Fourier analysis or wavelet analysis.

13. A storage medium readable on a computer, which stores a program wherein the method according to any one of claims 1 to 10 is constructed using a mathematical means, the program realizing, on a computer, a function capable of predicting novel biological•functional and/or binding activity of desired protein, amino acid sequence, or nucleotide sequence.

14. A binding mode of at least two kinds of arbitrary proteins (or amino acid sequences, nucleotide sequences), which is predicted by the method according to any one of claims 1 to 10.

15. An application of biological•functional activity of an arbitrary protein or nucleotide sequence predicted by the method according to any one of claims 1 to 10,
the activity being employed for at least one selected from a pesticide as a therapeutic agent, a medicament as a therapeutic agent, prevention of a hereditary disease, diagnosis of a hereditary disease, prevention of a pestilence, and diagnosis of a pestilence.
